(19) United States
(12) Patent Application Publication
(10) Pub. No.: US 2013/0066063 A1
(43) Pub. Date: Mar. 14, 2013

(54) BICYCLO[6.1.0]NON-4-YNE REGENTS FOR CHEMICAL MODIFICATION OF OLIGONUCLEOTIDES

(71) Applicants: John Cooke Hodges, Ann Arbor, MI (US); Floris Louis van Delft, Nijmegen (NL); Sander Sebastiaan Van Berkel, Lent (NL); Jorge Verkade, Nijmegen (NL); Lana L. Berry, Ann Arbor, MI (US)

(72) Inventors: David A. Berry, Ann Arbor, MI (US); John Cooke Hodges, Ann Arbor, MI (US); Floris Louis van Delft, Nijmegen (NL); Sander Sebastiaan Van Berkel, Lent (NL); Jorge Verkade, Nijmegen (NL)

(21) Appl. No.: 13/606,560

(22) Filed: Sep. 7, 2012

Related U.S. Application Data
(60) Provisional application No. 61/532,867, filed on Sep. 9, 2011.
(60) Provisional application No. 61/613,599, filed on Mar. 21, 2012.

(51) Int. Cl. C07F 9/06; C07H 19/048

(52) U.S. Cl. 536/26.8; 558/386

(57) ABSTRACT

The present invention provides for compounds of Formulae I and II:

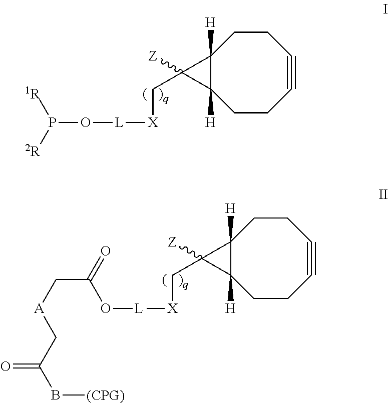

wherein ¹R, ²R, L, X, q, Z, A, and B have any of the values disclosed in the specification. Compounds of Formulae I and II are useful as reagents to introduce bicyclo[6.1.0]non-4-yne groups into oligonucleotide chains to serve as points of attachment for chemical tags.

### CROSS-REFERENCE TO RELATED APPLICATIONS

This application claims the benefit of the filing date under 35 U.S.C. §119(e) of U.S. Provisional Patent Application Ser. No. 61/532,867, filed Sep. 9, 2011, and U.S. Provisional Patent Application Ser. No. 61/613,599, filed Mar. 21, 2012, both of which are hereby incorporated by reference.

### BACKGROUND

Those skilled in the art of chemical synthesis of single stranded segments of nucleic acids (also known as “oligonucleotides” or “oligos”) frequently wish to introduce chemical tags that facilitate the scientific study of DNA and RNA. Examples of chemical tags that are frequently employed for this purpose include fluorescent dyes, quencher dyes, luminescent compounds, biotin, desthiobiotin, antigens, enzyme cofactors, heavy isotopes, radioactive isotopes, and the like.

One highly flexible approach to introduce the chemical tag to biological macromolecules such as proteins, enzymes, antibodies, oligosaccharides, and oligonucleotides is through a two step process involving a “click” reaction (1). In the first step, an alkyne is incorporated into the macromolecule. In the second step, a chemical tag that has been modified to contain an azide group is introduced, inducing a click reaction that covalently links the macromolecule and the chemical tag via their alkyne and azide functionalities, respectively (Scheme 1). A major advantage of the click reaction approach is that a single preparation of alkyne-modified macromolecule may be divided into portions and each portion may be coupled with a different azide-modified tag. This provides an efficient means of scientific exploration of a biological macromolecule of interest since a single lot of alkyne-modified macromolecule can be tagged with a wide variety of tags using a process that is simple and offers nearly a quantitative yield.

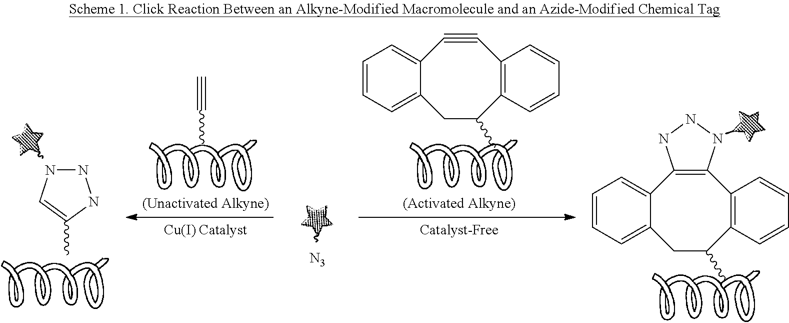

Click reactions between azide and alkyne counterparts fall into two categories (Scheme 1). The first category is the metal catalyzed click reaction, wherein a terminal alkyne and an azide react in the presence of a metal catalyst, usually a Cu(I) species (copper-catalyzed azide-alkyne cycloaddition or CuAAC). The second category is the catalyst-free click reaction, wherein an alkyne that has enhanced activity in the click reaction is used.

In the case of oligonucleotides, it is especially advantageous to employ a catalyst-free click reaction as a means of introducing chemical tags since there are numerous sites on the oligonucleotide that can coordinate with Cu(I), reducing its ability to catalyze triazole formation and causing partial degradation of the oligonucleotide (2). To this end, reagents have been developed for incorporating a dibenzocyclooctyne (DIBO) moiety into an oligonucleotide (3-5). The resulting DIBO-containing oligonucleotides undergo catalyst-free click reactions with azides. However, additional reagents for the incorporation of activated alkynes into oligonucleotides are needed. Bicyclononane (BCN) is known to undergo catalyst-free click reactions (6-7), but reagents for incorporating BCN into an oligonucleotide have not been reported.

### BACKGROUND REFERENCES

(1) U.S. Pat. No. 7,375,234, Copper-Catalysed Ligation of Azides and Acetylenes, May 20, 2008.
(2) Kanan, M. W.; Rozenman, M. M.; Sakurai, K.; Snyder, T. M.; Liu, D. R., Nature, 2004, 431, 545-549.
(3) Marks, I. S.; Kand, J. S.; Jones, B. T.; Landmark, K. J.; Cleland, A. J.; Taton, T. A., Bioconjugate Chemistry, 2011, 22, 1259-1263.
(4) Jayaprakash, K. N.; Peng, C. G.; Butler, D.; Varghese, J. P.; Maier, M. A.; Rajeev, K. G.; Manoharan, M., Organic Letters, 2010, 12(23), 5410-13.
(5) van Delft, P.; Meeuwenoord, N. J.; Hoogendoorn, S.; Dinkelaar, J.; Overkleeft, H. S.; van der Marel, G. A.; Filippov, D. V., Organic Letters, 2010, 12(23), 5486-5489.
(6) Dommerholt, J.; Schmidt, S.; Temming, R.; Hendriks, L. J. A.; Rutjes, F. P. J. T.; van Hest, J. C. M.; Lefeber, D. J.; Friedl, P.; van Delft, F. L., Angewande Chemie, International Edition, 2010, 49, 9422-9425.
(7) PCT/NL2011/050280, Fused Cyclooctyne Compounds and their Use in Metal-Free Click Reactions, unpublished.

### SUMMARY OF INVENTION

This invention provides phosphoramidite bicyclo[6.1.0]non-4-yne reagents and solid-supported bicyclo[6.1.0]non-4-yne reagents. These reagents are compatible with the current state of the art for the chemical synthesis of DNA oligonucleotides and RNA oligonucleotides. These reagents may be used to incorporate reactive alkyne moieties in synthetic oligonucleotides, thereby enabling further derivatization of the oligo via catalyst-free click reactions with azide-containing tags.

The first aspect of this invention is a phosphorous (III)-containing compound of Formula I:

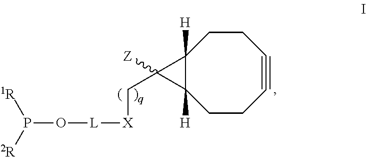

wherein:
q is 1, 2, or 3;
R¹— and R²— are independently N≡CCH₂CH₂O—, (C₁-C₆ alkyl)O—, or (C₁-C₆ alkyl)₂N—;
Z— is H—, or DMT-OCH₂—;
—X— and -L- are either both absent or both present;
—X— is absent or is —O—, —NH—, —S—, —NHCO₂—, —O₂CNH—, —NHCONH—, —NHCSNH—, —CONH—, —CO₂—, or —OCO₂—; and
-L- is absent or is selected from a group consisting of —(CH₂)ₙ—, —(CH₂CH₂O)ₙ(CH₂)ₘ—, —(CH₂CH₂CH₂O)ₙ(CH₂)ₘ—, —(CH₂)₃S₂(CH₂)₃—, —(CH₂)₆S₂(CH₂)₆—, —(CH₂)₂—O—(CH₂)₃S₂(CH₂)₃—O—(CH₂)₂—, —CH(CH₂O-DMT)CH₂—, —CH(CH₂O-DMT)CH₂—O—(CH₂)ₘ—, —CH(CH₂CH₂O-DMT)CH₂CH₂—, —CH(CH₂CH₂O-DMT)CH₂CH₂—O—(CH₂)ₘ—,

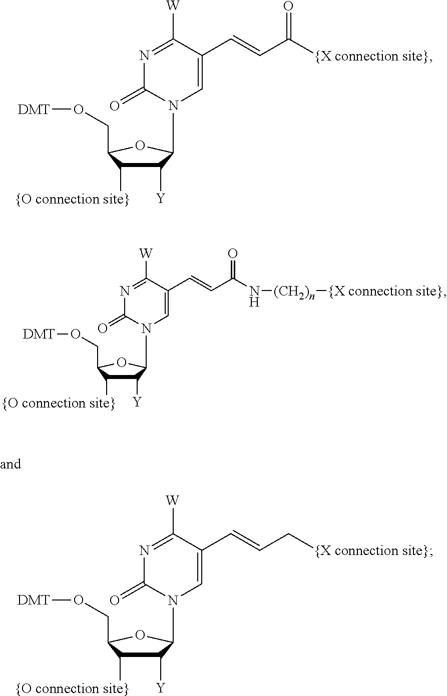

wherein n is 2-6, m is 2-3, Y is H, O-TBS, O—POM, or O-TOM, and W is OH, N═CHN(CH₃)₂, NHCOPh, or NHCOCH₃.

Any of the compounds of Formula I may be employed when a BCN group is to be installed at the 5′-terminus of an oligo. Those reagents of Formula I, wherein -L- or Z— includes a DMTO-moiety may also be employed when a BCN group is to be installed at an internal sequence position of an oligo.

The second aspect of the invention is a solid-supported compound of Formula II:

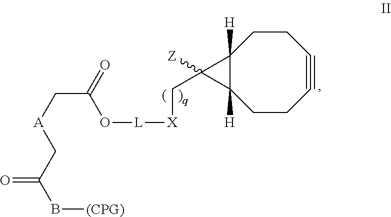

wherein:
q is 1, 2, or 3;
-A- is absent or is —O— or —O—(C₆H₄)—O—;
—B— is Icaa or aminopropyl;
Z— is H—, DMT-OCH₂—, or HOCH₂—;
—X— and -L- are either both present or both absent;
—X— is absent or is —O—, —NH—, —S—, —NHCO₂—, —O₂CNH—, —NHCONH—, —NHCSNH—, —CONH—, —CO₂—, or —OCO₂—; and
-L- is absent or is selected from a group consisting of —CH(CH₂O-DMT)CH₂—, —CH(CH₂O-DMT)CH₂—O—(CH₂)ₘ—, —CH(CH₂CH₂O-DMT)CH₂CH₂—, —CH(CH₂CH₂O-DMT)CH₂CH₂—O—(CH₂)ₘ—, —CH(CH₂OH)CH₂—, —CH(CH₂OH)CH₂O(CH₂)ₘ—, —CH(CH₂CH₂OH)CH₂CH₂—, —CH(CH₂CH₂OH)CH₂CH₂O(CH₂)ₘ—.

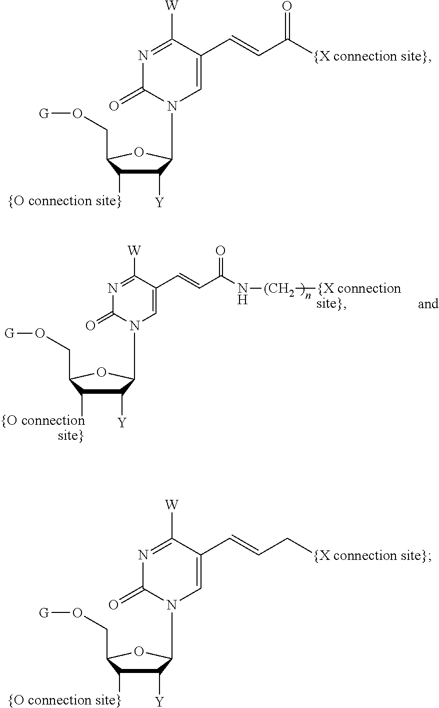

wherein n is 2-6, m is 2-3, Y is H, O-TBS, O—POM, or O-TOM, G is DMT or H, and W is OH, N═CHN(CH₃)₂, NHCOPh, or NHCOCH₃;
wherein —X— and -L- are present when Z— is H.

Any of the compounds of Formula II may be employed when a BCN group is to be installed at the 3′-terminus of an oligo.

### DETAILED DESCRIPTION

Wherever used within this specification, the following definitions and abbreviations apply:

“Alkyl” refers to a saturated hydrocarbon group, and includes saturated hydrocarbon groups in which the carbon atoms are arranged in a linear, branched, or cyclic fashion, or combinations thereof. “C₁-C₆ alkyl” refers to an alkyl group having one to six carbon atoms.

“BCN” is bicyclo[6.1.0]non-4-yne.

“CPG” is controlled pore glass.

“DMT” is dimethoxytrityl.

“DNA” is deoxyribonucleic acid.

“HPLC” is high performance liquid chromatography.

“Icaa” is long chain aminoalkyl, a linker that is commonly applied to controlled pore glass, the combination of which forms an insoluble solid support that is well known to those skilled in the art of DNA and RNA synthesis as lcaa-CPG.

“Mesylate” is methanesulfonate.

“MMT” is monomethoxytrityl.

“Oligo” is a shortened term for oligonucleotide.

“POM” is pivaloyloxymethyl.

“Ph” is phenyl.

“RNA” is ribonucleic acid.

“TBS” is tert-butyl-dimethylsilyl.

“TOM” is tri-iso-propylsilyloxymethyl.

### PREFERRED EMBODIMENTS OF THE COMPOUNDS OF THE INVENTION

In some embodiments, the invention includes the following compounds of Formula I:
(1) compounds wherein R¹ is N≡CCH₂CH₂O— and R² is (i-Pr)₂N—;
(2) compounds wherein Z is
   (a) H—; or
   (b) DMT-OCH₂—;
(3) compounds wherein —X— is
   (a) —O—, —NH—, or —NHCO₂— or is absent;
   (b) —O—, —NH—, —S—, —NHCO₂—, or —NHCONH—;
   (c) —O—, —NH—, or —S—;
   (d) —NHCO₂—, or —NHCONH—;
   (e) —NHCO₂—; or
   (f) —O—, —NH—, —S—, —NHCO₂—, —O₂CNH—, —NHCONH—, —NHCSNH—, or —CONH—, or is absent;
(4) compounds wherein -L- is
   (a) —(CH₂)ₙ—, —(CH₂CH₂O)ₙ(Ch₂)ₘ—, or —(CH₂CH₂CH₂O)ₙ(CH₂)ₘ—, or is absent;
   (b) —(CH₂)₃S₂(CH₂)₃—, —(CH₂)₆S₂(CH₂)₆—, or —(CH₂)₂—O—(CH₂)₃S₂(CH₂)₃O(CH₂)₂—;
   (c) —(CH₂)ₙ—, —(CH₂CH₂O)ₙ(CH₂)ₘ—, —(CH₂)₃S₂(CH₂)₃—, —CH(CH₂O-DMT)CH₂O(CH₂)ₘ—, —CH(CH₂CH₂O-DMT)CH₂CH₂O(CH₂)ₘ—,

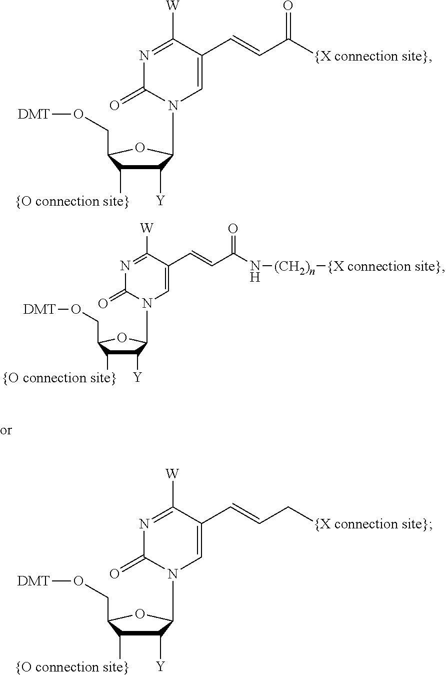

(d) —CH(CH₂O-DMT)CH₂O(CH₂)ₘ— or —CH(CH₂CH₂O-DMT)CH₂CH₂O(CH₂)ₘ—;

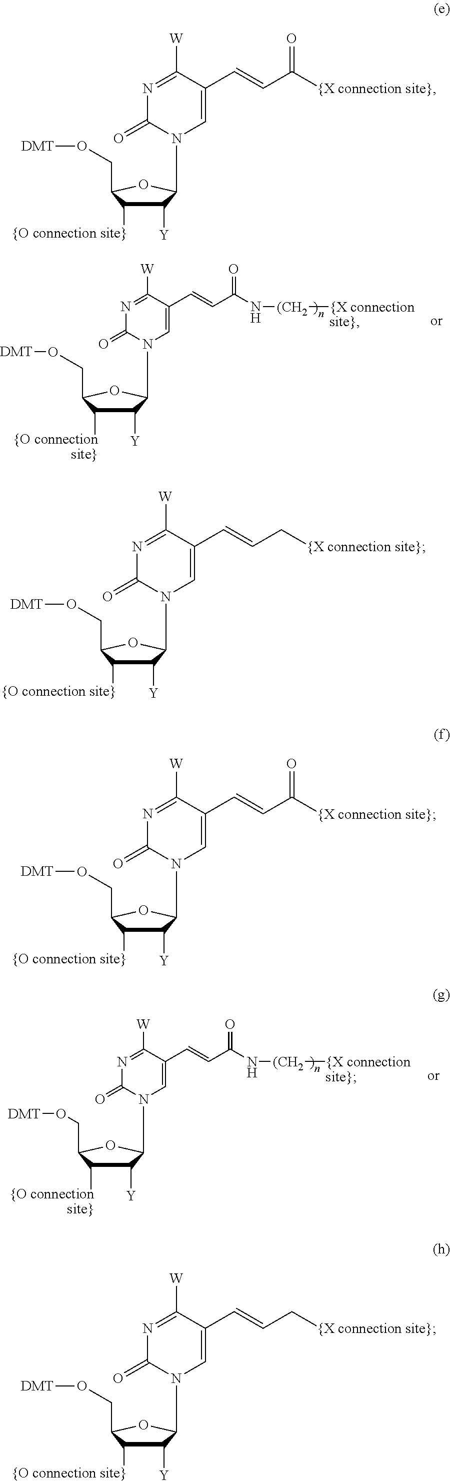

(5) compounds wherein -L- is absent or is selected from a group consisting of —(CH₂)ₙ—, —(CH₂CH₂O)ₙ(CH₂)ₘ—, —(CH₂CH₂CH₂O)ₙ(CH₂)ₘ—, —(CH₂)₃S₂(CH₂)₃—, —(CH₂)₆S₂(CH₂)₆—, and —(CH₂)₂—O—(CH₂)₃S₂(CH₂)₃—O—(CH₂)₂— when Z— is DMT-OCH₂—;
(6) compounds wherein —X— and -L- are both present when q is 1;
(7) compounds wherein q is 1;
(8) compounds wherein q is 2;
(9) compounds wherein q is 3; and
(10) the compounds listed in Table 1.

It is understood that the invention also includes compounds of Formula I having any combination of the attributes listed in (1) through (9) above. For example, further embodiments of the invention can be obtained by combining (1), (2)(a), (3)(a), (4)(a), and (6); (1), (2)(a), (3)(a), (4)(b), and (6); (1), (2)(a), (3)(a), (4)(c), and (6); (1), (2)(a), (3)(a), (4)(d), and (6); (1), (2)(a), (3)(a), (4)(e), and (6); (1), (2)(a), (3)(a), (4)(f), and (6); (1), (2)(a), (3)(a), (4)(g), and (6); (1), (2)(a), (3)(a), (4)(h), and (6); (1), (2)(a), (3)(b), (4)(a), and (6); (1), (2)(a), (3)(b), (4)(b), and (6); (1), (2)(a), (3)(b), (4)(c), and (6); (1), (2)(a), (3)(b), (4)(d), and (6); (1), (2)(a), (3)(b), (4)(e), and (6); (1), (2)(a), (3)(b), (4)(f), and (6); (1), (2)(a), (3)(b), (4)(g), and (6); (1), (2)(a), (3)(b), (4)(h), and (6); (1), (2)(a), (3)(c), (4)(a), and (6); (1), (2)(a), (3)(c), (4)(b), and (6); (1), (2)(a), (3)(c), (4)(c), and (6); (1), (2)(a), (3)(c), (4)(d), and (6); (1), (2)(a), (3)(c), (4)(e), and (6); (1), (2)(a), (3)(c), (4)(f), and (6); (1), (2)(a), (3)(c), (4)(g), and (6); (1), (2)(a), (3)(c), (4)(h), and (6); (1), (2)(a), (3)(d), (4)(a), and (6); (1), (2)(a), (3)(d), (4)(b), and (6); (1), (2)(a), (3)(d), (4)(c), and (6); (1), (2)(a), (3)(d), (4)(d), and (6); (1), (2)(a), (3)(d), (4)(e), and (6); (1), (2)(a), (3)(d), (4)(f), and (6); (1), (2)(a), (3)(d), (4)(g), and (6); (1), (2)(a), (3)(d), (4)(h), and (6); (1), (2)(a), (3)(e), (4)(a), and (6); (1), (2)(a), (3)(e), (4)(b), and (6); (1), (2)(a), (3)(e), (4)(c), and (6); (1), (2)(a), (3)(e), (4)(d), and (6); (1), (2)(a), (3)(e), (4)(e), and (6); (1), (2)(a), (3)(e), (4)(f), and (6); (1), (2)(a), (3)(e), (4)(g), and (6); (1), (2)(a), (3)(e), (4)(h), and (6); (1), (2)(a), (3)(f), (4)(a), and (6); (1), (2)(a), (3)(f), (4)(b), and (6); (1), (2)(a), (3)(f), (4)(c), and (6); (1), (2)(a), (3)(f), (4)(d), and (6); (1), (2)(a), (3)(f), (4)(e), and (6); (1), (2)(a), (3)(f), (4)(f), and (6); (1), (2)(a), (3)(f), (4)(g), and (6); (1), (2)(a), (3)(f), (4)(h), and (6); (1), (2)(b), (3)(a), (4)(a), and (6); (1), (2)(b), (3)(a), (4)(b), and (6); (1), (2)(b), (3)(a), (4)(c), and (6); (1), (2)(b), (3)(a), (4)(d), and (6); (1), (2)(b), (3)(a), (4)(e), and (6); (1), (2)(b), (3)(a), (4)(f), and (6); (1), (2)(b), (3)(a), (4)(g), and (6); (1), (2)(b), (3)(a), (4)(h), and (6); (1), (2)(b), (3)(b), (4)(a), and (6); (1), (2)(b), (3)(b), (4)(b), and (6); (1), (2)(b), (3)(b), (4)(c), and (6); (1), (2)(b), (3)(b), (4)(d), and (6); (1), (2)(b), (3)(b), (4)(e), and (6); (1), (2)(b), (3)(b), (4)(f), and (6); (1), (2)(b), (3)(b), (4)(g), and (6); (1), (2)(b), (3)(b), (4)(h), and (6); (1), (2)(b), (3)(c), (4)(a), and (6); (1), (2)(b), (3)(c), (4)(b), and (6); (1), (2)(b), (3)(c), (4)(c), and (6); (1), (2)(b), (3)(c), (4)(d), and (6); (1), (2)(b), (3)(c), (4)(e), and (6); (1), (2)(b), (3)(c), (4)(f), and (6); (1), (2)(b), (3)(c), (4)(g), and (6); (1), (2)(b), (3)(c), (4)(h), and (6); (1), (2)(b), (3)(d), (4)(a), and (6); (1), (2)(b), (3)(d), (4)(b), and (6); (1), (2)(b), (3)(d), (4)(c), and (6); (1), (2)(b), (3)(d), (4)(d), and (6); (1), (2)(b), (3)(d), (4)(e), and (6); (1), (2)(b), (3)(d), (4)(f), and (6); (1), (2)(b), (3)(d), (4)(g), and (6); (1), (2)(b), (3)(d), (4)(h), and (6); (1), (2)(b), (3)(e), (4)(a), and (6); (1), (2)(b), (3)(e), (4)(b), and (6); (1), (2)(b), (3)(e), (4)(c), and (6); (1), (2)(b), (3)(e), (4)(d), and (6); (1), (2)(b), (3)(e), (4)(e), and (6); (1), (2)(b), (3)(e), (4)(f), and (6); (1), (2)(b), (3)(e), (4)(g), and (6); (1), (2)(b), (3)(e), (4)(h), and (6); (1), (2)(b), (3)(f), (4)(a), and (6); (1), (2)(b), (3)(f), (4)(b), and (6); (1), (2)(b), (3)(f), (4)(c), and (6); (1), (2)(b), (3)(f), (4)(d), and (6); (1), (2)(b), (3)(f), (4)(e), and (6); (1), (2)(b), (3)(f), (4)(f), and (6); (1), (2)(b), (3)(f), (4)(g), and (6); (1), (2)(b), (3)(f), (4)(h), and (6); (5) and (6); (1), (2)(a), (3)(a), (4)(a), and (7); (1), (2)(a), (3)(a), (4)(b), and (7); (1), (2)(a), (3)(a), (4)(c), and (7); (1), (2)(a), (3)(a), (4)(d), and (7); (1), (2)(a), (3)(a), (4)(e), and (7); (1), (2)(a), (3)(a), (4)(f), and (7); (1), (2)(a), (3)(a), (4)(g), and (7); (1), (2)(a), (3)(a), (4)(h), and (7); (1), (2)(a), (3)(b), (4)(a), and (7); (1), (2)(a), (3)(b), (4)(b), and (7); (1), (2)(a), (3)(b), (4)(c), and (7); (1), (2)(a), (3)(b), (4)(d), and (7); (1), (2)(a), (3)(b), (4)(e), and (7); (1), (2)(a), (3)(b), (4)(f), and (7); (1), (2)(a), (3)(b), (4)(g), and (7); (1), (2)(a), (3)(b), (4)(h), and (7); (1), (2)(a), (3)(c), (4)(a), and (7); (1), (2)(a), (3)(c), (4)(b), and (7); (1), (2)(a), (3)(c), (4)(c), and (7); (1), (2)(a), (3)(c), (4)(d), and (7); (1), (2)(a), (3)(c), (4)(e), and (7); (1), (2)(a), (3)(c), (4)(f), and (7); (1), (2)(a), (3)(c), (4)(g), and (7); (1), (2)(a), (3)(c), (4)(h), and (7); (1), (2)(a), (3)(d), (4)(a), and (7); (1), (2)(a), (3)(d), (4)(b), and (7); (1), (2)(a), (3)(d), (4)(c), and (7); (1), (2)(a), (3)(d), (4)(d), and (7); (1), (2)(a), (3)(d), (4)(e), and (7); (1), (2)(a), (3)(d), (4)(f), and (7); (1), (2)(a), (3)(d), (4)(g), and (7); (1), (2)(a), (3)(d), (4)(h), and (7); (1), (2)(a), (3)(e), (4)(a), and (7); (1), (2)(a), (3)(e), (4)(b), and (7); (1), (2)(a), (3)(e), (4)(c), and (7); (1), (2)(a), (3)(e), (4)(d), and (7); (1), (2)(a), (3)(e), (4)(e), and (7); (1), (2)(a), (3)(e), (4)(f), and (7); (1), (2)(a), (3)(e), (4)(g), and (7); (1), (2)(a), (3)(e), (4)(h), and (7); (1), (2)(a), (3)(f), (4)(a), and (7); (1), (2)(a), (3)(f), (4)(b), and (7); (1), (2)(a), (3)(f), (4)(c), and (7); (1), (2)(a), (3)(f), (4)(d), and (7); (1), (2)(a), (3)(f), (4)(e), and (7); (1), (2)(a), (3)(f), (4)(f), and (7); (1), (2)(a), (3)(f), (4)(g), and (7); (1), (2)(a), (3)(f), (4)(h), and (7); (1), (2)(b), (3)(a), (4)(a), and (7); (1), (2)(b), (3)(a), (4)(b), and (7); (1), (2)(b), (3)(a), (4)(c), and (7); (1), (2)(b), (3)(a), (4)(d), and (7); (1), (2)(b), (3)(a), (4)(e), and (7); (1), (2)(b), (3)(a), (4)(f), and (7); (1), (2)(b), (3)(a), (4)(g), and (7); (1), (2)(b), (3)(a), (4)(h), and (7); (1), (2)(b), (3)(b), (4)(a), and (7); (1), (2)(b), (3)(b), (4)(b), and (7); (1), (2)(b), (3)(b), (4)(c), and (7); (1), (2)(b), (3)(b), (4)(d), and (7); (1), (2)(b), (3)(b), (4)(e), and (7); (1), (2)(b), (3)(b), (4)(f), and (7); (1), (2)(b), (3)(b), (4)(g), and (7); (1), (2)(b), (3)(b), (4)(h), and (7); (1), (2)(b), (3)(c), (4)(a), and (7); (1), (2)(b), (3)(c), (4)(b), and (7); (1), (2)(b), (3)(c), (4)(c), and (7); (1), (2)(b), (3)(c), (4)(d), and (7); (1), (2)(b), (3)(c), (4)(e), and (7); (1), (2)(b), (3)(c), (4)(f), and (7); (1), (2)(b), (3)(c), (4)(g), and (7); (1), (2)(b), (3)(c), (4)(h), and (7); (1), (2)(b), (3)(d), (4)(a), and (7); (1), (2)(b), (3)(d), (4)(b), and (7); (1), (2)(b), (3)(d), (4)(c), and (7); (1), (2)(b), (3)(d), (4)(d), and (7); (1), (2)(b), (3)(d), (4)(e), and (7); (1), (2)(b), (3)(d), (4)(f), and (7); (1), (2)(b), (3)(d), (4)(g), and (7); (1), (2)(b), (3)(d), (4)(h), and (7); (1), (2)(b), (3)(e), (4)(a), and (7); (1), (2)(b), (3)(e), (4)(b), and (7); (1), (2)(b), (3)(e), (4)(c), and (7); (1), (2)(b), (3)(e), (4)(d), and (7); (1), (2)(b), (3)(e), (4)(e), and (7); (1), (2)(b), (3)(e), (4)(f), and (7); (1), (2)(b), (3)(e), (4)(g), and (7); (1), (2)(b), (3)(e), (4)(h), and (7); (1), (2)(b), (3)(f), (4)(a), and (7); (1), (2)(b), (3)(f), (4)(b), and (7); (1), (2)(b), (3)(f), (4)(c), and (7); (1), (2)(b), (3)(f), (4)(d), and (7); (1), (2)(b), (3)(f), (4)(e), and (7); (1), (2)(b), (3)(f), (4)(f), and (7); (1), (2)(b), (3)(f), (4)(g), and (7); (1), (2)(b), (3)(f), (4)(h), and (7); (5) and (7); (6) and (7); (1), (2)(a), (3)(a), (4)(a), and (8); (1), (2)(a), (3)(a), (4)(b), and (8); (1), (2)(a), (3)(a), (4)(c), and (8); (1), (2)(a), (3)(a), (4)(d), and (8); (1), (2)(a), (3)(a), (4)(e), and (8); (1), (2)(a), (3)(a), (4)(f), and (8); (1), (2)(a), (3)(a), (4)(g), and (8); (1), (2)(a), (3)(a), (4)(h), and (8); (1), (2)(a), (3)(b), (4)(a), and (8); (1), (2)(a), (3)(b), (4)(b), and (8); (1), (2)(a), (3)(b), (4)(c), and (8); (1), (2)(a), (3)(b), (4)(d), and (8); (1), (2)(a), (3)(b), (4)(e), and (8); (1), (2)(a), (3)(b), (4)(f), and (8); (1), (2)(a), (3)(b), (4)(g), and (8); (1), (2)(a), (3)(b), (4)(h), and (8); (1), (2)(a), (3)(c), (4)(a), and (8); (1), (2)(a), (3)(c), (4)(b), and (8); (1), (2)(a), (3)(c), (4)(c), and (8); (1), (2)(a), (3)(c), (4)(d), and (8); (1), (2)(a), (3)(c), (4)(e), and (8); (1), (2)(a), (3)(c), (4)(f), and (8); (1), (2)(a), (3)(c), (4)(g), and (8); (1), (2)(a), (3)(c), (4)(h), and (8); (1), (2)(a), (3)(d), (4)(a), and (8); (1), (2)(a), (3)(d), (4)(b), and (8); (1), (2)(a), (3)(d), (4)(c), and (8); (1), (2)(a), (3)(d), (4)(d), and (8); (1), (2)(a), (3)(d), (4)(e), and (8); (1), (2)(a), (3)(d), (4)(f), and (8); (1), (2)(a), (3)(d), (4)(g), and (8); (1), (2)(a), (3)(d), (4)(h), and (8); (1), (2)(a), (3)(e), (4)(a), and (8); (1), (2)(a), (3)(e), (4)(b), and (8); (1), (2)(a), (3)(e), (4)(c), and (8); (1), (2)(a), (3)(e), (4)(d), and (8); (1), (2)(a), (3)(e), (4)(e), and (8); (1), (2)(a), (3)(e), (4)(f), and (8); (1), (2)(a), (3)(e), (4)(g), and (8); (1), (2)(a), (3)(e), (4)(h), and (8); (1), (2)(a), (3)(f), (4)(a), and (8); (1), (2)(a), (3)(f), (4)(b), and (8); (1), (2)(a), (3)(f), (4)(c), and (8); (1), (2)(a), (3)(f), (4)(d), and (8); (1), (2)(a), (3)(f), (4)(e), and (8); (1), (2)(a), (3)(f), (4)(f), and (8); (1), (2)(a), (3)(f), (4)(g), and (8); (1), (2)(a), (3)(f), (4)(h), and (8); (1), (2)(b), (3)(a), (4)(a), and (8); (1), (2)(b), (3)(a), (4)(b), and (8); (1), (2)(b), (3)(a), (4)(c), and (8); (1), (2)(b), (3)(a), (4)(d), and (8); (1), (2)(b), (3)(a), (4)(e), and (8); (1), (2)(b), (3)(a), (4)(f), and (8); (1), (2)(b), (3)(a), (4)(g), and (8); (1), (2)(b), (3)(a), (4)(h), and (8); (1), (2)(b), (3)(b), (4)(a), and (8); (1), (2)(b), (3)(b), (4)(b), and (8); (1), (2)(b), (3)(b), (4)(c), and (8); (1), (2)(b), (3)(b), (4)(d), and (8); (1), (2)(b), (3)(b), (4)(e), and (8); (1), (2)(b), (3)(b), (4)(f), and (8); (1), (2)(b), (3)(b), (4)(g), and (8); (1), (2)(b), (3)(b), (4)(h), and (8); (1), (2)(b), (3)(c), (4)(a), and (8); (1), (2)(b), (3)(c), (4)(b), and (8); (1), (2)(b), (3)(c), (4)(c), and (8); (1), (2)(b), (3)(c), (4)(d), and (8); (1), (2)(b), (3)(c), (4)(e), and (8); (1), (2)(b), (3)(c), (4)(f), and (8); (1), (2)(b), (3)(c), (4)(g), and (8); (1), (2)(b), (3)(c), (4)(h), and (8); (1), (2)(b), (3)(d), (4)(a), and (8); (1), (2)(b), (3)(d), (4)(b), and (8); (1), (2)(b), (3)(d), (4)(c), and (8); (1), (2)(b), (3)(d), (4)(d), and (8); (1), (2)(b), (3)(d), (4)(e), and (8); (1), (2)(b), (3)(d), (4)(f), and (8); (1), (2)(b), (3)(d), (4)(g), and (8); (1), (2)(b), (3)(d), (4)(h), and (8); (1), (2)(b), (3)(e), (4)(a), and (8); (1), (2)(b), (3)(e), (4)(b), and (8); (1), (2)(b), (3)(e), (4)(c), and (8); (1), (2)(b), (3)(e), (4)(d), and (8); (1), (2)(b), (3)(e), (4)(e), and (8); (1), (2)(b), (3)(e), (4)(f), and (8); (1), (2)(b), (3)(e), (4)(g), and (8); (1), (2)(b), (3)(e), (4)(h), and (8); (1), (2)(b), (3)(f), (4)(a), and (8); (1), (2)(b), (3)(f), (4)(b), and (8); (1), (2)(b), (3)(f), (4)(c), and (8); (1), (2)(b), (3)(f), (4)(d), and (8); (1), (2)(b), (3)(f), (4)(e), and (8); (1), (2)(b), (3)(f), (4)(f), and (8); (1), (2)(b), (3)(f), (4)(g), and (8); (1), (2)(b), (3)(f), (4)(h), and (8); (5) and (8); (6) and (8); (1), (2)(a), (3)(a), (4)(a), and (9); (1), (2)(a), (3)(a), (4)(b), and (9); (1), (2)(a), (3)(a), (4)(c), and (9); (1), (2)(a), (3)(a), (4)(d), and (9); (1), (2)(a), (3)(a), (4)(e), and (9); (1), (2)(a), (3)(a), (4)(f), and (9); (1), (2)(a), (3)(a), (4)(g), and (9); (1), (2)(a), (3)(a), (4)(h), and (9); (1), (2)(a), (3)(b), (4)(a), and (9); (1), (2)(a), (3)(b), (4)(b), and (9); (1), (2)(a), (3)(b), (4)(c), and (9); (1), (2)(a), (3)(b), (4)(d), and (9); (1), (2)(a), (3)(b), (4)(e), and (9); (1), (2)(a), (3)(b), (4)(f), and (9); (1), (2)(a), (3)(b), (4)(g), and (9); (1), (2)(a), (3)(b), (4)(h), and (9); (1), (2)(a), (3)(c), (4)(a), and (9); (1), (2)(a), (3)(c), (4)(b), and (9); (1), (2)(a), (3)(c), (4)(c), and (9); (1), (2)(a), (3)(c), (4)(d), and (9); (1), (2)(a), (3)(c), (4)(e), and (9); (1), (2)(a), (3)(c), (4)(f), and (9); (1), (2)(a), (3)(c), (4)(g), and (9); (1), (2)(a), (3)(c), (4)(h), and (9); (1), (2)(a), (3)(d), (4)(a), and (9); (1), (2)(a), (3)(d), (4)(b), and (9); (1), (2)(a), (3)(d), (4)(c), and (9); (1), (2)(a), (3)(d), (4)(d), and (9); (1), (2)(a), (3)(d), (4)(e), and (9); (1), (2)(a), (3)(d), (4)(f), and (9); (1), (2)(a), (3)(d), (4)(g), and (9); (1), (2)(a), (3)(d), (4)(h), and (9); (1), (2)(a), (3)(e), (4)(a), and (9); (1), (2)(a), (3)(e), (4)(b), and (9); (1), (2)(a), (3)(e), (4)(c), and (9); (1), (2)(a), (3)(e), (4)(d), and (9); (1), (2)(a), (3)(e), (4)(e), and (9); (1), (2)(a), (3)(e), (4)(f), and (9); (1), (2)(a), (3)(e), (4)(g), and (9); (1), (2)(a), (3)(e), (4)(h), and (9); (1), (2)(a), (3)(f), (4)(a), and (9); (1), (2)(a), (3)(f), (4)(b), and (9); (1), (2)(a), (3)(f), (4)(c), and (9); (1), (2)(a), (3)(f), (4)(d), and (9); (1), (2)(a), (3)(f), (4)(e), and (9); (1), (2)(a), (3)(f), (4)(f), and (9); (1), (2)(a), (3)(f), (4)(g), and (9); (1), (2)(a), (3)(f), (4)(h), and (9); (1), (2)(b), (3)(a), (4)(a), and (9); (1), (2)(b), (3)(a), (4)(b), and (9); (1), (2)(b), (3)(a), (4)(c), and (9); (1), (2)(b), (3)(a), (4)(d), and (9); (1), (2)(b), (3)(a), (4)(e), and (9); (1), (2)(b), (3)(a), (4)(f), and (9); (1), (2)(b), (3)(a), (4)(g), and (9); (1), (2)(b), (3)(a), (4)(h), and (9); (1), (2)(b), (3)(b), (4)(a), and (9); (1), (2)(b), (3)(b), (4)(b), and (9); (1), (2)(b), (3)(b), (4)(c), and (9); (1), (2)(b), (3)(b), (4)(d), and (9); (1), (2)(b), (3)(b), (4)(e), and (9); (1), (2)(b), (3)(b), (4)(f), and (9); (1), (2)(b), (3)(b), (4)(g), and (9); (1), (2)(b), (3)(b), (4)(h), and (9); (1), (2)(b), (3)(c), (4)(a), and (9); (1), (2)(b), (3)(c), (4)(b), and (9); (1), (2)(b), (3)(c), (4)(c), and (9); (1), (2)(b), (3)(c), (4)(d), and (9); (1), (2)(b), (3)(c), (4)(e), and (9); (1), (2)(b), (3)(c), (4)(f), and (9); (1), (2)(b), (3)(c), (4)(g), and (9); (1), (2)(b), (3)(c), (4)(h), and (9); (1), (2)(b), (3)(d), (4)(a), and (9); (1), (2)(b), (3)(d), (4)(b), and (9); (1), (2)(b), (3)(d), (4)(c), and (9); (1), (2)(b), (3)(d), (4)(d), and (9); (1), (2)(b), (3)(d), (4)(e), and (9); (1), (2)(b), (3)(d), (4)(f), and (9); (1), (2)(b), (3)(d), (4)(g), and (9); (1), (2)(b), (3)(d), (4)(h), and (9); (1), (2)(b), (3)(e), (4)(a), and (9); (1), (2)(b), (3)(e), (4)(b), and (9); (1), (2)(b), (3)(e), (4)(c), and (9); (1), (2)(b), (3)(e), (4)(d), and (9); (1), (2)(b), (3)(e), (4)(e), and (9); (1), (2)(b), (3)(e), (4)(f), and (9); (1), (2)(b), (3)(e), (4)(g), and (9); (1), (2)(b), (3)(e), (4)(h), and (9); (1), (2)(b), (3)(f), (4)(a), and (9); (1), (2)(b), (3)(f), (4)(b), and (9); (1), (2)(b), (3)(f), (4)(c), and (9); (1), (2)(b), (3)(f), (4)(d), and (9); (1), (2)(b), (3)(f), (4)(e), and (9); (1), (2)(b), (3)(f), (4)(f), and (9); (1), (2)(b), (3)(f), (4)(g), and (9); (1), (2)(b), (3)(f), (4)(h), and (9); (5) and (9); (6) and (9); and the like.

As is readily apparent to those skilled in the art, the compounds of formula I may exist in more than one tautomeric form known as “tautomers.” For example, thymine may exist as either the 5-methylpyrimidine-2,4(1H,3H)-dione tautomer or the 4-hydroxy-5-methylpyrimidin-2(1H)-one tautomer. Where tautomers exist, each tautomeric form, and mixtures thereof, are contemplated as included in the present invention. When any reference in this application to one of the specific tautomers of the compounds of formula I is given, it is understood to encompass every tautomeric form and mixtures thereof.

TABLE 1
Compounds of Formula I

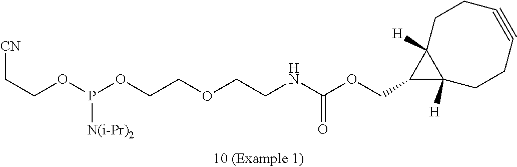

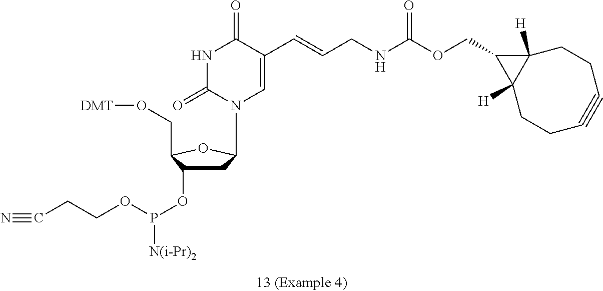

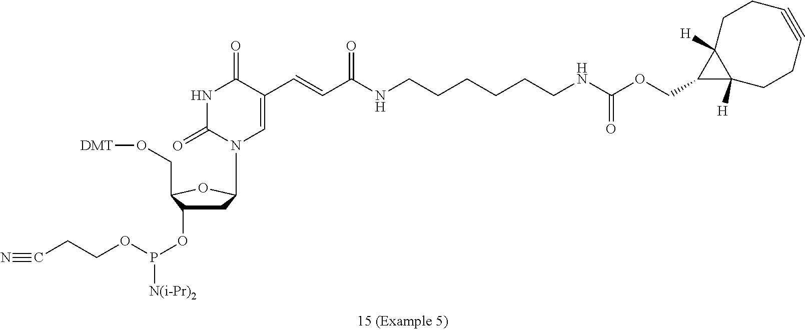

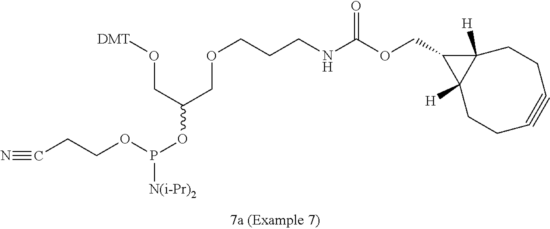

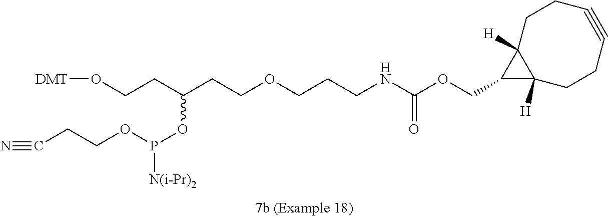

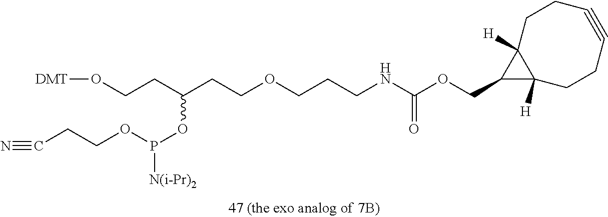

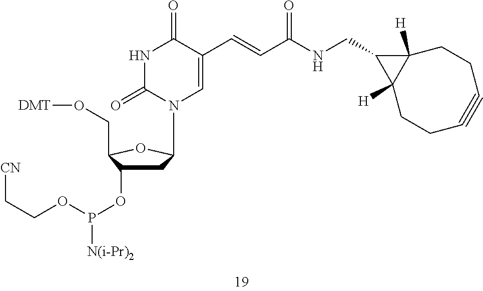

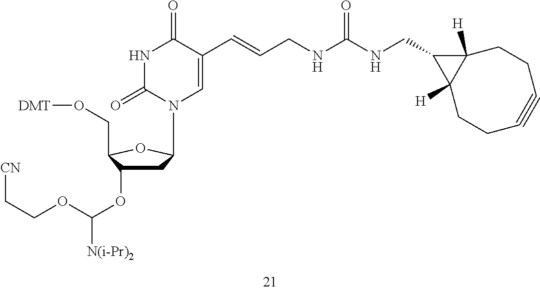

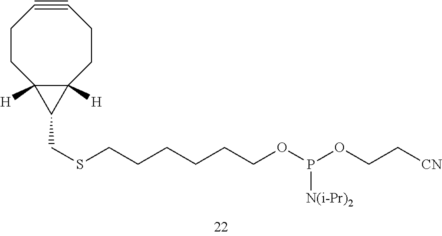

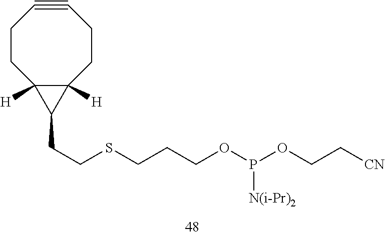

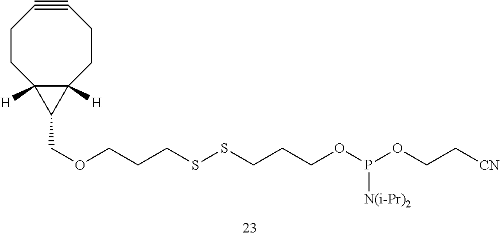

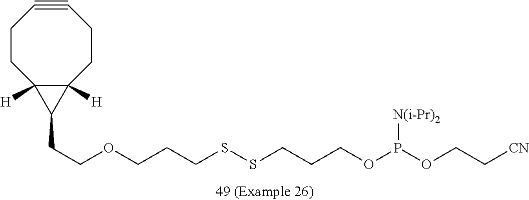

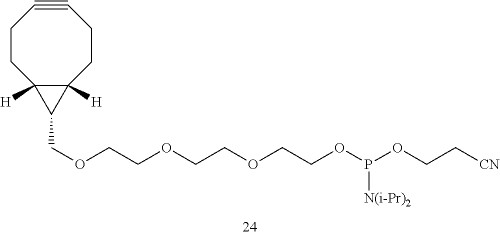

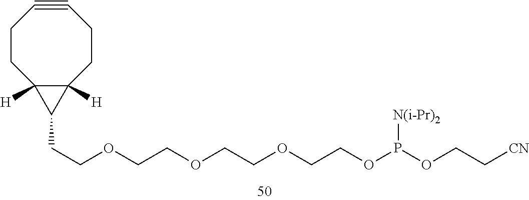

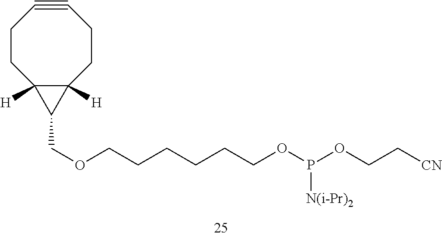

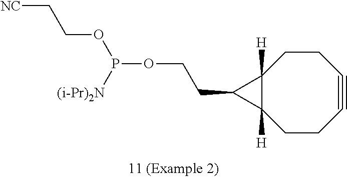

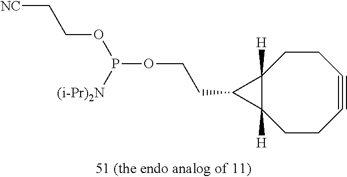

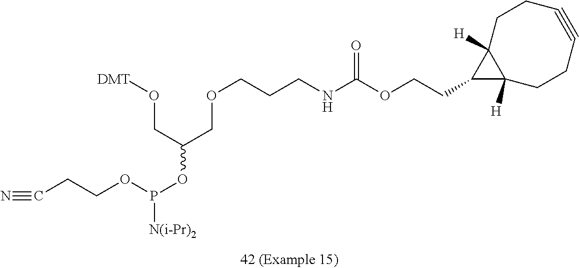

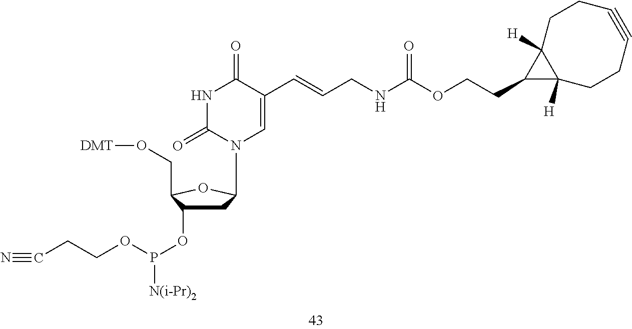

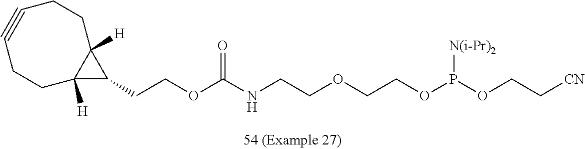

In some embodiments, the invention includes the following compounds of Formula II:

(1) compounds wherein -A- is absent or is —O—;

(2) compounds wherein —B— is Icaa;

(3) compounds wherein Z is
(a) H—; or
(b) DMT-OCH₂;

(4) compounds wherein —X— is
(a) —O—, —NH—, —S—, or —NHCO₂—;
(b) —NHCO₂—, or —NHCONH—;
(c) —NHCO₂—; or
(d) —O—, —NH—, —S—, —NHCO₂—, —O₂CNH—, —NHCONH—, —NHCSNH—, or —CONH—, or is absent;

(5) compounds wherein -L- is
(a) —CH(CH₂O-DMT)CH₂—, —CH(CH₂O-DMT)CH₂O(CH₂)ₘ—, —CH(CH₂CH₂O-DMT)CH₂CH₂—, or —CH(CH₂CH₂O-DMT)CH₂CH₂—O—(CH₂)ₘ—;
(b) —CH(CH₂OH)CH₂—, —CH(CH₂OH)CH₂O(CH₂)ₘ—, or —CH(CH₂CH₂OH)CH₂CH₂—, —CH(CH₂CH₂OH)CH₂CH₂O(CH₂)ₘ—;

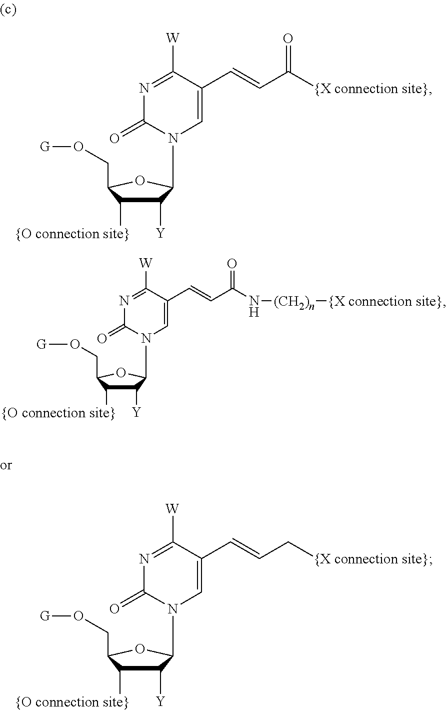

(d) —CH(CH₂O-DMT)CH₂—, —CH(CH₂O-DMT)CH₂O(CH₂)ₘ—, —CH(CH₂CH₂O-DMT)CH₂CH₂—, —CH(CH₂CH₂O-DMT)CH₂CH₂O(CH₂)ₘ—,

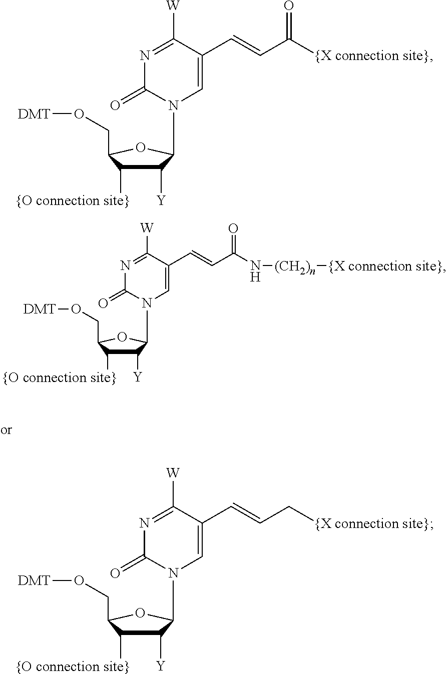

(6) compounds wherein —X— and -L- are absent when Z— is DMT-OCH₂— or HOCH₂—;

(7) compounds wherein q is 1;

(8) compounds wherein q is 2

(9) compounds wherein q is 3; and

(10) the compounds listed in Table 2.

It is understood that the invention also includes compounds of Formula II having any combination of the attributes listed in (1) through (9) above. For example, further embodiments of the invention can be obtained by combining (1), (2), (3)(a), (4)(a), (5)(a), and (7); (1), (2), (3)(a), (4)(a), (5)(b), and (7); (1), (2), (3)(a), (4)(a), (5)(c), and (7); (1), (2), (3)(a), (4)(a), (5)(d), and (7); (1), (2), (3)(a), (4)(b), (5)(a), and (7); (1), (2), (3)(a), (4)(b), (5)(b), and (7); (1), (2), (3)(a), (4)(b), (5)(c), and (7); (1), (2), (3)(a), (4)(b), (5)(d), and (7); (1), (2), (3)(a), (4)(c), (5)(a), and (7); (1), (2), (3)(a), (4)(c), (5)(b), and (7); (1), (2), (3)(a), (4)(c), (5)(c), and (7); (1), (2), (3)(a), (4)(c), (5)(d), and (7); (1), (2), (3)(a), (4)(d), (5)(a), and (7); (1), (2), (3)(a), (4)(d), (5)(b), and (7); (1), (2), (3)(a), (4)(d), (5)(c), and (7); (1), (2), (3)(a), (4)(d), (5)(d), and (7); (1), (2), (3)(b), and (4)(a); (1), (2), (3)(b), and (4)(b); (1), (2), (3)(b), and (4)(c); (1), (2), (3)(b), and (4)(d); (6) and (7); (1), (2), (3)(a), (4)(a), (5)(a), and (8); (1), (2), (3)(a), (4)(a), (5)(b), and (8); (1), (2), (3)(a), (4)(a), (5)(c), and (8); (1), (2), (3)(a), (4)(a), (5)(d), and (8); (1), (2), (3)(a), (4)(b), (5)(a), and (8); (1), (2), (3)(a), (4)(b), (5)(b), and (8); (1), (2), (3)(a), (4)(b), (5)(c), and (8); (1), (2), (3)(a), (4)(b), (5)(d), and (8); (1), (2), (3)(a), (4)(c), (5)(a), and (8); (1), (2), (3)(a), (4)(c), (5)(b), and (8); (1), (2), (3)(a), (4)(c), (5)(c), and (8); (1), (2), (3)(a), (4)(c), (5)(d), and (8); (1), (2), (3)(a), (4)(d), (5)(a), and (8); (1), (2), (3)(a), (4)(d), (5)(b), and (8); (1), (2), (3)(a), (4)(d), (5)(c), and (8); (1), (2), (3)(a), (4)(d), (5)(d), and (8); (6) and (8); (1), (2), (3)(a), (4)(a), (5)(a), and (9); (1), (2), (3)(a), (4)(a), (5)(b), and (9); (1), (2), (3)(a), (4)(a), (5)(c), and (9); (1), (2), (3)(a), (4)(a), (5)(d), and (9); (1), (2), (3)(a), (4)(b), (5)(a), and (9); (1), (2), (3)(a), (4)(b), (5)(b), and (9); (1), (2), (3)(a), (4)(b), (5)(c), and (9); (1), (2), (3)(a), (4)(b), (5)(d), and (9); (1), (2), (3)(a), (4)(c), (5)(a), and (9); (1), (2), (3)(a), (4)(c), (5)(b), and (9); (1), (2), (3)(a), (4)(c), (5)(c), and (9); (1), (2), (3)(a), (4)(c), (5)(d), and (9); (1), (2), (3)(a), (4)(d), (5)(a), and (9); (1), (2), (3)(a), (4)(d), (5)(b), and (9); (1), (2), (3)(a), (4)(d), (5)(c), and (9); (1), (2), (3)(a), (4)(d), (5)(d), and (9); (6) and (9); and the like.

TABLE 2
Compounds of Formula II

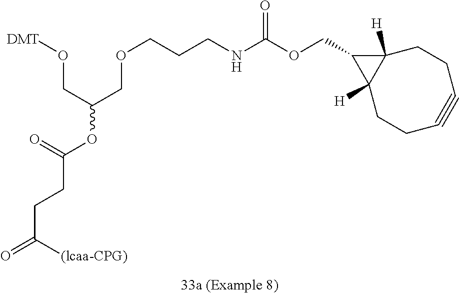

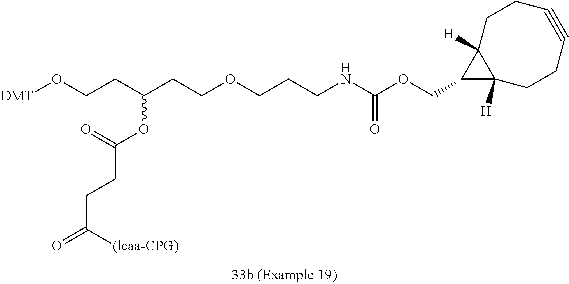

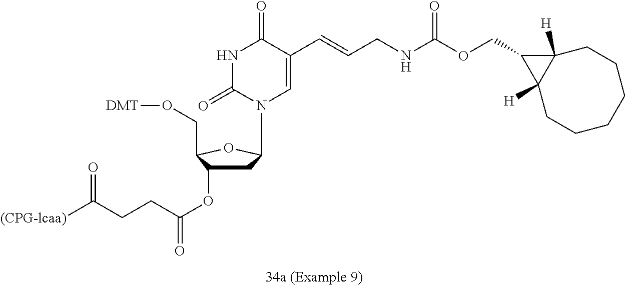

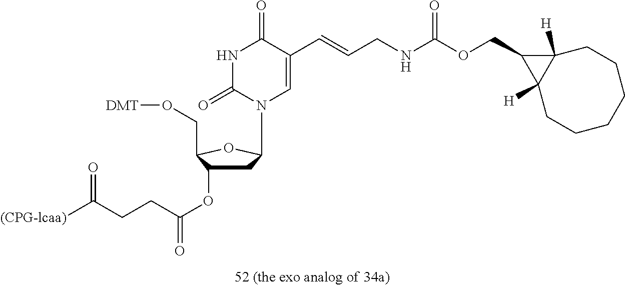

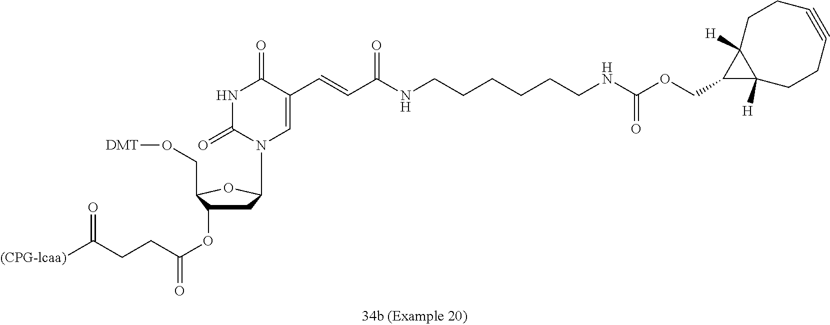

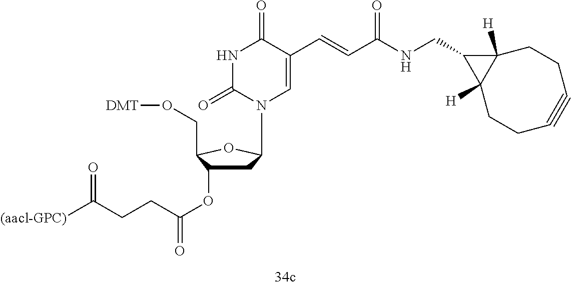

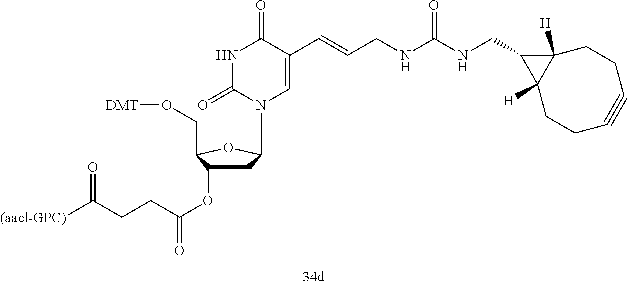

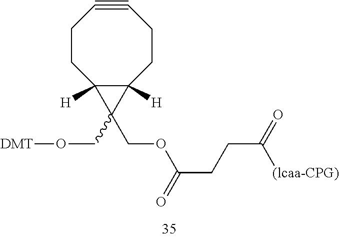

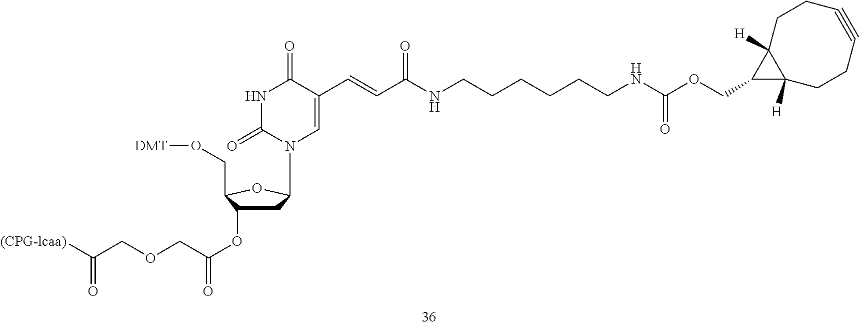

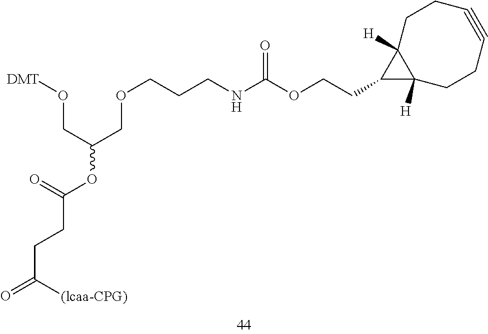

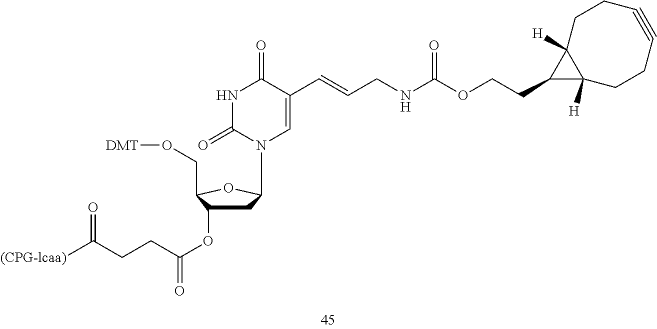

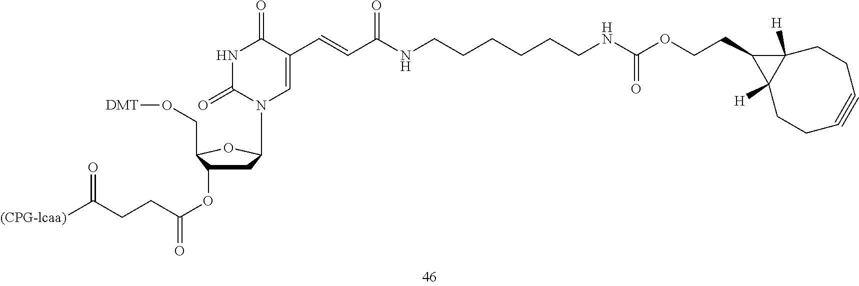

Methods of Preparation of Compounds of Formulae I and II

The compounds of Formulae I and II may be prepared from known compounds using synthetic transformations familiar to those having ordinary skill in the art. More specifically, each of the compounds of Formulae I and II may be prepared from BCN-methanol (vi(a)), DMT-OCH₂-BCN-methanol (vi(b)), BCN-ethanol (vi(c)), BCN-propanol (vi(d)), DMT-OCH₂-BCN-ethanol (vi(e)), or DMT-OCH₂-BCN-propanol (vi(t)), which all may be prepared from 1,5-octadiene as shown in Scheme 2.

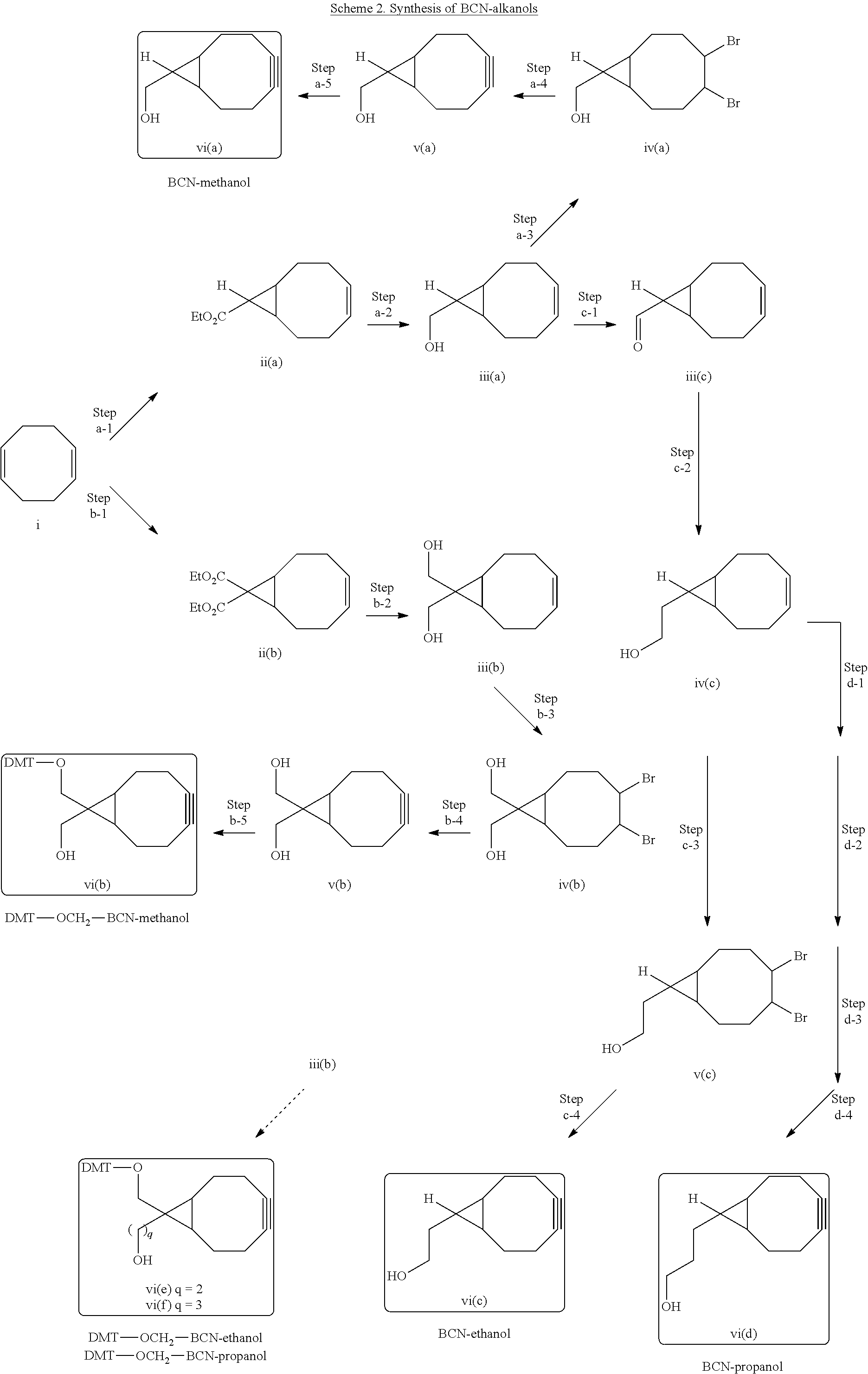

The synthesis of vi(a) from 1,5-octadiene, as illustrated in Scheme 2, has been described previously by Dommerholt et al., Angewande Chemie, International Edition, 2010, 49, 9422-9425, the entire contents of which are incorporated herein by reference. The synthesis of vi(b) proceeds in analogous fashion. The syntheses of vi(c) and vi(d) proceed via single application (Steps c-1 and c-2) or double application (Steps c-1 and c-2 followed by Steps d-1 and d-2) of the Wittig one-carbon homologation method. Subsequent conversion of the cyclooctenes to cyclooctynes is achieved through analogous bromination and dehydrobromination reactions. Detailed procedures for the syntheses of vi(b-d) are found in the Examples below. It is recognized by those with ordinary skill in the art of organic synthesis that DMT protection of one of the alcohol groups in iii(b) affords a suitable synthetic intermediate that may likewise undergo one or two Wittig one-carbon homologation(s) and analogous transformation of the ring double bond to a triple bond to provide DMT-OCH₂-BCN-ethanol (vi(e)) and DMT-OCH₂-BCN-propanol (vi(t)).

While both known diastereomers of BCN-methanol, the exo and endo isomers (Scheme 3), readily undergo Cu-free click reactions at room temperature, the reaction of the endo isomer of BCN-methanol is slightly faster than that of the exo isomer of BCN-methanol. Thus, the endo isomer of compounds of Formulae I and II wherein q is 1 may be preferred for applications where a fast reaction rate is vital. With BCN-ethanol and BCN-propanol, both exo and endo isomers undergo Cu-free click reactions at comparable rates. In the practice of chemical tagging of synthetic oligonucleotides, both endo and exo isomers of all compounds of Formulae I and II are generally sufficiently reactive to be useful. For simplicity of experimental design and ease of interpretation of results, it is preferable to choose one or the other and not work with a mixture of both isomers, however the use of a mixture of endo- and exo- isomers is chemically feasible.

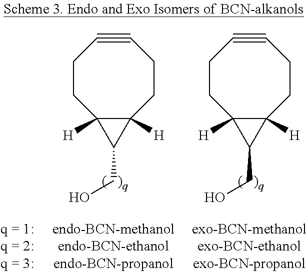

The compounds of Formulae I and II may be prepared from vi(a-t) according to the methods illustrated in Schemes 4a, 4b, 4c, 5a, and 5b. The procedures set forth in these schemes may be carried out with the endo isomer and/or the exo isomer of vi(a-t). Therefore, and for the sake of clarity, the specific isomers of vi(a-t), subsequent synthetic intermediates, and compounds of Formulae I and II are not specified in the schemes.

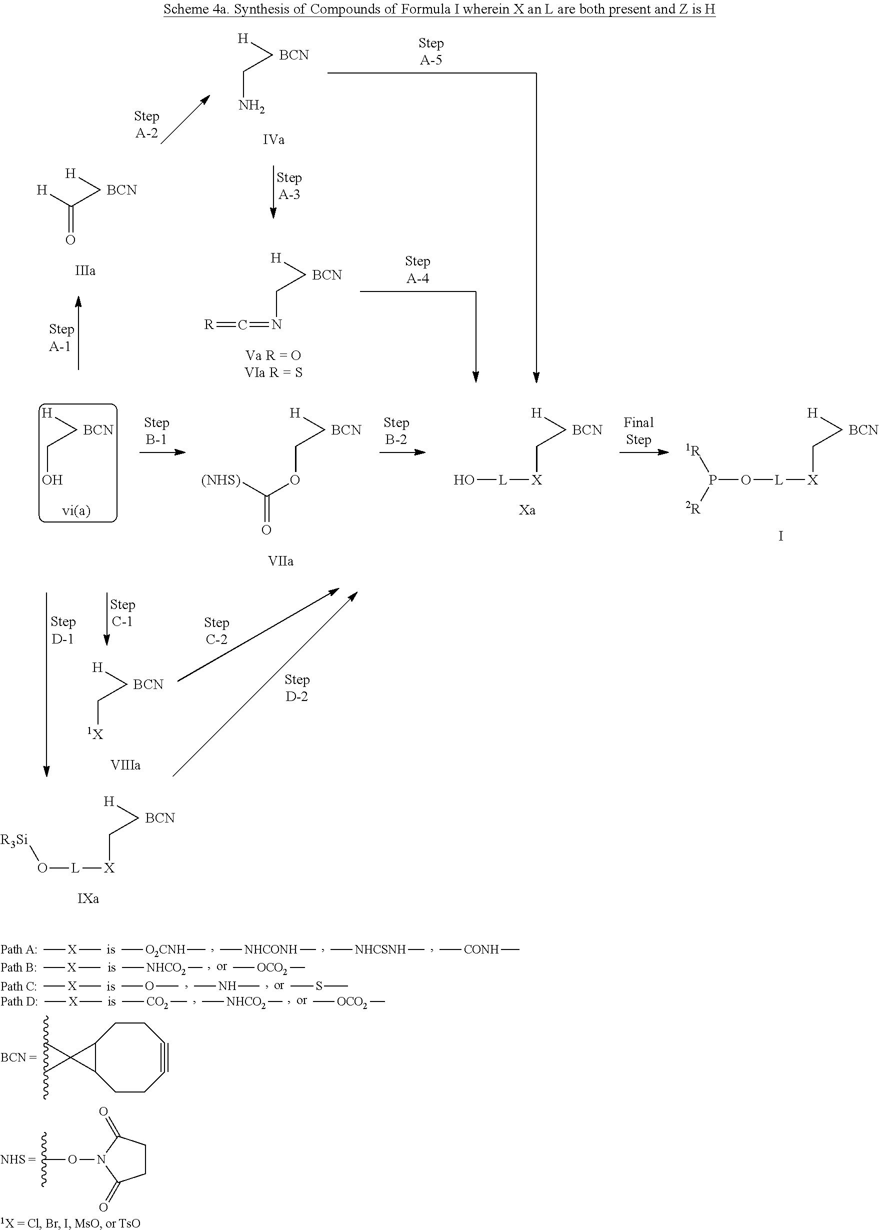

Scheme 4a illustrates the synthesis of the compounds of Formula Xa when X and L are both present, and Z is H. When —X— is —O₂CNH—, —NHCONH—, —NHCSNH—, or —CONH—, the compounds of Formula Xa may be prepared from BCN-methanol (vi(a)) in three or four steps via Path A. In Step A-1, vi(a) is converted to the aldehyde, IIIa, using oxidation procedures known to those with ordinary skill in the art of organic synthesis, such as Swern oxidation, Dess-Martin oxidation, Ley oxidation, chromium-based oxidations, and the like. Subsequently in Step A-2, treatment of 111a with ammonia and a hydride reducing agent such as NaCNBH₃, NaBH₄, or Na(OAc)₃H, gives the amine, IVa. This amine is a versatile synthetic intermediate, which may be converted in Step A-3 to the isocyanate, Va, by treatment with phosgene, or a phosgene equivalent, such as triphosgene, phenyl-chloroformate, 4-nitrophenyl-chloroformate, di-(2-pyridyl)-carbonate, or carbonyl-diimidazole, and a tertiary amine, such as N-methylmorpholine, triethylamine, or diisopropylethylamine. Similarly, IVa may be converted in Step A-3 to the isothiocyanate, VIa, by treatment with thiophosgene, or a thiophosgene equivalent such as thiocarbonyldiimidazole or di-(2-pyridyl)-thiocarbonate, and a tertiary amine such as N-methylmorpholine, triethylamine, or diisopropylethylamine. In Step A-4, treatment of Va or VIa with a primary amine affords compounds of formula Xa wherein —X— is —NHCONH— or —NHCSNH—, respectively. Alternatively in Step A-4, treatment of Va with an alcohol and a tertiary amine affords compounds of Formula Xa wherein —X— is —O₂CNH—. Furthermore, IVa may be directly acylated by an active ester, a succinimidylcarbonate, an isocyanate, or an isothiocyanate, and the like, in Step A-5, to give compounds of Formula Xa, wherein —X— is —CONH—, —O₂CNH—, —NHCONH—, or —NHCSNH—. Strategies for the inclusion of additional protection and deprotection steps in order to control the desired regio-chemical outcome in the steps depicted for Path A are familiar to those having ordinary skill in the art.

When —X— is —NHCO₂— or —OCO₂—, the compounds of Formula Xa may be prepared from BCN-methanol (vi(a)) in two steps via Path B. In Step B-1, vi(a) is treated with N,N′-disuccinimidyl carbonate, to afford VIIa according to the previously disclosed method of Dommerholt et al., Angewande Chemie, International Edition, 2010, 49, 9422-9425. Subsequent treatment with a primary amine or an alcohol and a tertiary amine such as N-methylmorpholine, triethylamine, or diisopropylethylamine in Step B-2 affords a compound of Formula Xa. Strategies for the inclusion of additional protection and deprotection steps in order to control the desired regio-chemical outcome in the steps depicted for Path B are familiar to those having ordinary skill in the art.

When —X— is —O—, —NH—, or —S—, the compounds of Formula Xa may be prepared from BCN-methanol (vi(a)) in two steps via Path C. In Step C-1, the hydroxyl group of vi(a) is converted to a leaving group, ¹X—, such as a tosylate, a mesylate, an iodide, a bromide, or a chloride, to provide VIIIa. For example, treatment with a sulfonyl chloride or a sulfonic anhydride and an organic base such as pyridine, collidine, N-methylmorpholine, triethylamine, or diisopropylethylamine provides VIIIa wherein ¹X— is tosylate or mesylate. Alternatively, treatment with triphenylphosphine and a halogen reagent such as I₂, N-bromosuccinimide, carbon tetrabromide, N-chlorosuccinimide, or carbon tetrachloride provides VIIIa, wherein ¹X— is I, Br, or Cl. Displacement of the leaving group in Step C-2 by an O, N, or S nucleophile provides a compound of Formula Xa. Strategies for the inclusion of additional protection and deprotection steps in order to control the desired regio-chemical outcome in the steps depicted for Path C are familiar to those having ordinary skill in the art.

When —X— is —CO₂—, —NHCO₂— or —OCO₂—, the compounds of Formula Xa may be prepared from BCN-methanol (vi(a)) in two steps via Path D. In Step D-1, the hydroxyl group of vi(a) is acylated by a acylating reagent, such as a carboxylic acid chloride, a carboxylic acid anhydride, an active ester, an isocyanate, or a chloroformate, bearing a pendant silyl-protected hydroxyl group, in combination with an organic base such as pyridine, collidine, 4-dimethylaminopyridine, N-methylmorpholine, triethylamine, or diisopropylamine to provide IXa. Removal of the silyl protecting group by treatment with tetrabutylammonium floride, potassium fluoride, triethylammonium hydrofluoride, or pyridinium hydrofluoride affords a compound of Formula Xa. Strategies for the use of alternative protecting groups and deprotection conditions in Path D are familiar to those having ordinary skill in the art.

In the Final Step, the penultimate intermediate, Xa, which may be prepared by Path A, B, C or D, is converted to a compound of Formula I by treatment with a reactive phosphorous(III) reagent. For example, treatment of Xa with (i-Pr₂N)₂POCH₂CH₂CN, also known as “bis-reagent”, and an acid catalyst, such as tetrazole, provides a compound of Formula I in which R¹— is N≡CCH₂CH₂O— and R²— is (i-Pr)₂N. Similarly for example, treatment of Xa with i-Pr₂NP(Cl)OCH₂CH₂CN, also known as “chloro-reagent”, and a tertiary amine, such as diisopropylethylamine, provides also a compound of Formula I in which R¹— is N≡CCH₂CH₂O— and R²— is (i-Pr)₂N.

It is recognized by those with ordinary skill in the art of organic synthesis that BCN-ethanol (vi(c)) or BCN-propanol (vi(d)) may be substituted for BCN-methanol (vi(a)) as the starting material in Scheme 4a, thereby affording homologous intermediates of Formulae Xc and Xd, respectively, and homologous compounds of Formula I as the final product.

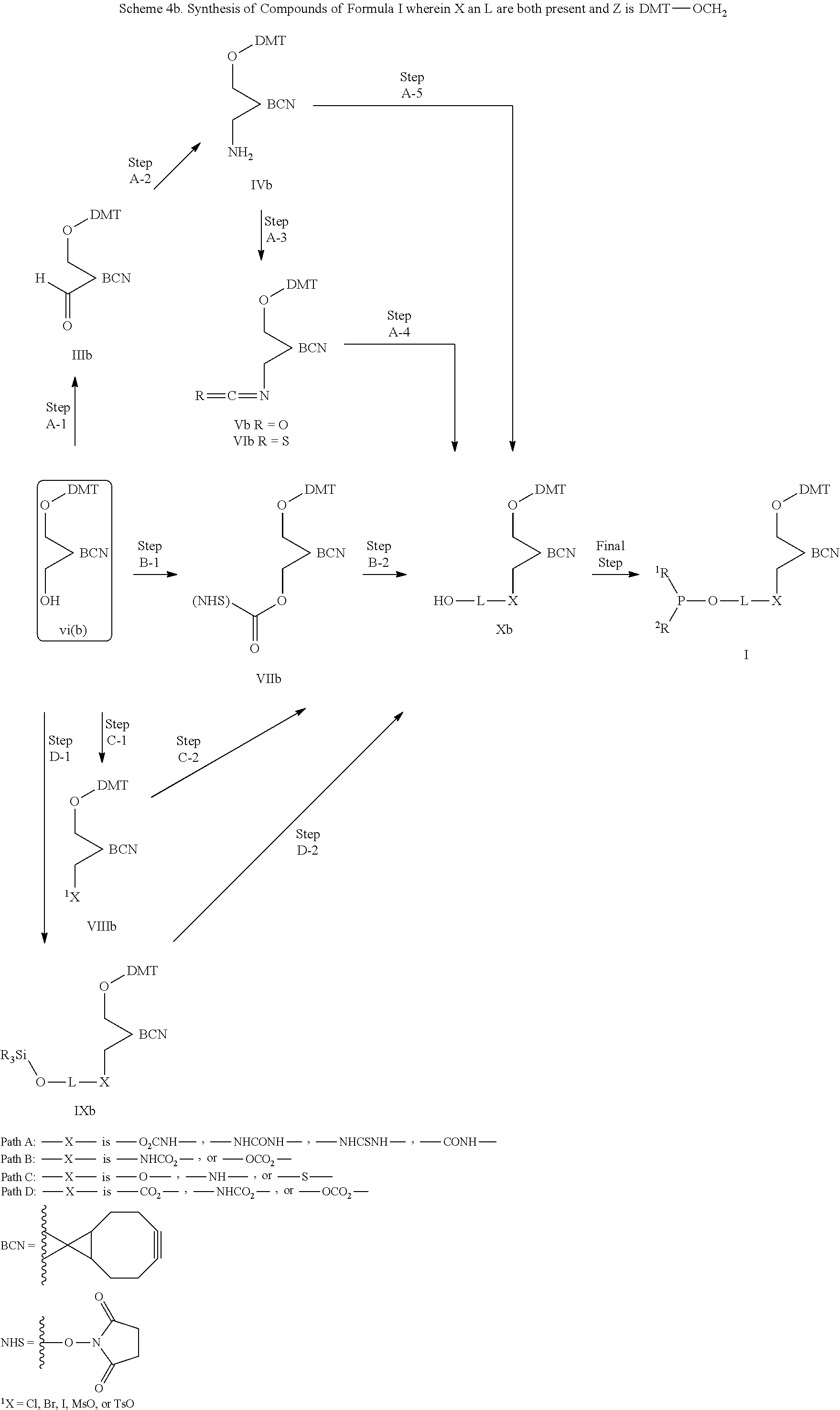

Scheme 4b illustrates the synthesis of the compounds of Formula I when X and L are both present, and Z is DMT-OCH₂. The synthetic transformations of Paths A, B, C, D as described above for Scheme 4a are analogously applicable to Scheme 4b. Thus, Paths A, B, C, and D convert DMT-OCH₂-BCN-methanol (vi(b)) to a compound of Formula Xb. Subsequently, Xb is transformed into a compound of Formula I in the Final Step. It is recognized by those with ordinary skill in the art of organic synthesis that DMT-OCH₂-BCN-ethanol (vi(e)) or DMT-OCH₂-BCN-propanol (vi(t)) may be substituted for DMT-OCH₂-BCN-methanol (vi(b)) as the starting material in Scheme 4b, thereby affording homologous intermediates of Formulae Xe and Xf, respectively, and homologous compounds of Formula I as the final product.

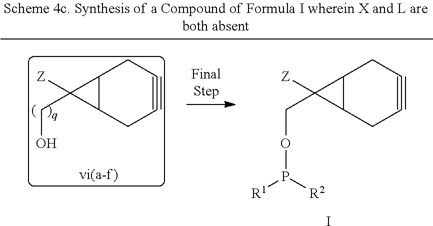

Scheme 4c illustrates the synthesis of a compound of Formula I from vi(a-f) when X and L are both absent. The starting alcohol, BCN-methanol (vi(a)), DMT-OCH₂-BCN-methanol (vi(b)), BCN-ethanol (vi(c)), DMT-OCH₂-BCN-ethanol (vi(e)), BCN-propanol (vi(d)), or DMT-OCH₂-BCN-propanol (vi(f)), is subjected to the conditions of the Final Step as described above for Schemes 4a and 4b.

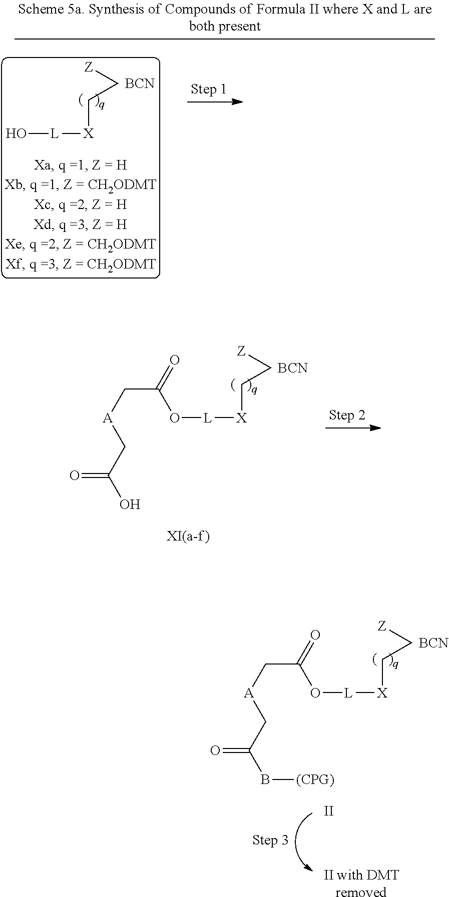

Scheme 5a illustrates the synthesis of the compounds of Formula II when X and L are both present. In Step 1, the penultimate synthetic intermediates, X(a-f), as synthesized in Schemes 4a and 4b, are employed. They are converted to a monoester of a dicarboxylic acid of the formula HO₂CCH₂ACH₂CO₂H, wherein A is absent, O, or O(C₆H₄)O. When A is absent, X(a-f) is treated with succinic anhydride and pyridine to give XI(a-f). When A is O, the dicarboxylic acid is treated with a carbodiimide reagent such as ethyl-dimethylaminoethylcarbodiimide (EDC) to generate a solution of the cyclic anhydride, which is reacted with X(a-f) in the presence of pyridine to provide XI(a-f). When A is O(C₆H₄)O, the dicarboxylic acid is treated with two molar equivalents of a carbodiimide reagent such as ethyl-dimethylaminoethylcarbodiimide and two equivalents of N-hydroxysuccinimide to generate a solution of the bis-NHS ester, which is reacted with X(a-f) in the presence of pyridine to provide XI(a-f) upon quenching the reaction with water. In order to avoid making the bis-ester, a molar excess of the dicarboxylic acid is used relative to X(a-f). In Step 1,4-dimethylaminopyridine (DMAP) may optionally be used to facilitate the formation of XI(a-f) under mild conditions. In Step 2, the carboxylic acid group of XI(a-f) is treated with one of a variety of amide bond coupling reagents that are well known to those skilled in the art. For example, treatment of XI(a-f) with PYBOP (benzotriazol-1-yl-oxy-tris-pyrrolidino-phosphonium hexafluorophosphate) and diisopropylethylamine, followed by addition of lcaa-CPG or aminopropyl-CPG affords a compound of Formula II. In step 3, additional compounds of Formula II are also provided by removal of the DMT group that is present, either in Z or in L by treatment with an organic acid, such as for example dichloroacetic acid, in a solvent, such as for example dichloromethane.

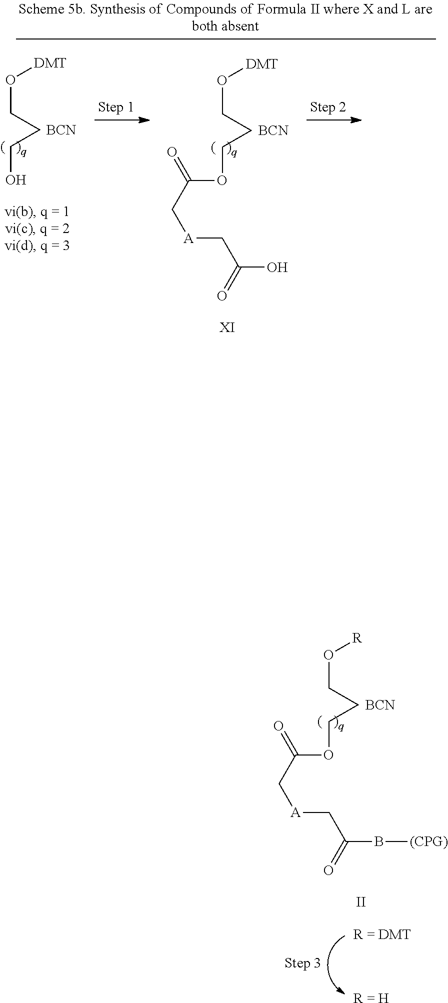

Scheme 5b illustrates the synthesis of the compounds of Formula II when X and L are both absent. Starting from the synthetic intermediates vi(b), vi(e), or vi(t) of Scheme 2, the synthetic transformations of Scheme 5a are analogously applied in Steps 1-3 of Scheme 5b to afford a compound of Formula II.

Methods of Incorporating BCN—Containing Compounds of Formulas I and II into Oligonucleotides

The BCN-containing compounds of Formulas I and II may be employed in automated DNA synthesis to introduce the BCN group into DNA oligonucleotides. As will be appreciated by those skilled in the art of DNA synthesis, such automated synthesis may be conducted as follows: (1) a desired DNA sequence is programmed into an automated DNA synthesizer that has been equipped with the necessary reagents; (2) the synthesizer carries out the synthesis steps in automated fashion over a number of cycles, adding each prescribed nucleotide of the sequence until the full-length, protected oligonucleotide is prepared on a solid support; and (3) the oligonucleotide is cleaved from the solid support, and protecting groups are removed, to give the free oligonucleotide.

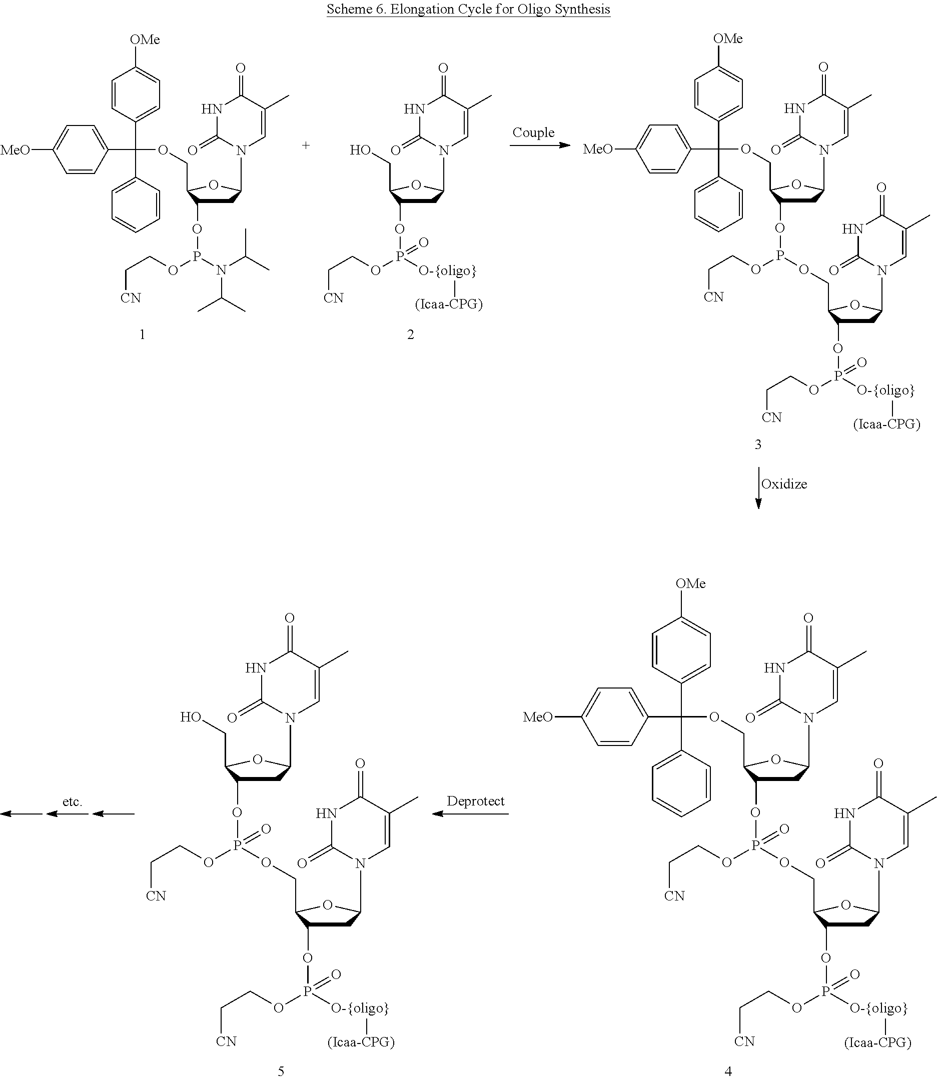

As shown in Scheme 6, the first nucleoside is attached to the solid support (e.g., CPG) via a cleavable linkage (e.g. Icaa or aminopropyl). The oligo is subsequently elongated using successive cycles of coupling, oxidation, and deprotection reactions. In the coupling reaction, the incoming nucleoside is in the form of its 5′-dimethoxytrityl-protected-3′-phosphoramidite, 1. The phosphoramidite provides a reactive P(III) group which efficiently couples with the terminal hydroxyl group on 2 via displacement of diisopropyl amine. The resulting phosphite linkage of 3 is then oxidized to a phosphate linkage of 4. Removal of the dimethoxytrityl (“DMT”) protecting group readies the oligo, 5, for the next incoming phosphoramidite. The full length oligo is cleaved from the support and all protecting groups are removed, affording an oligo of reasonable purity for many purposes.

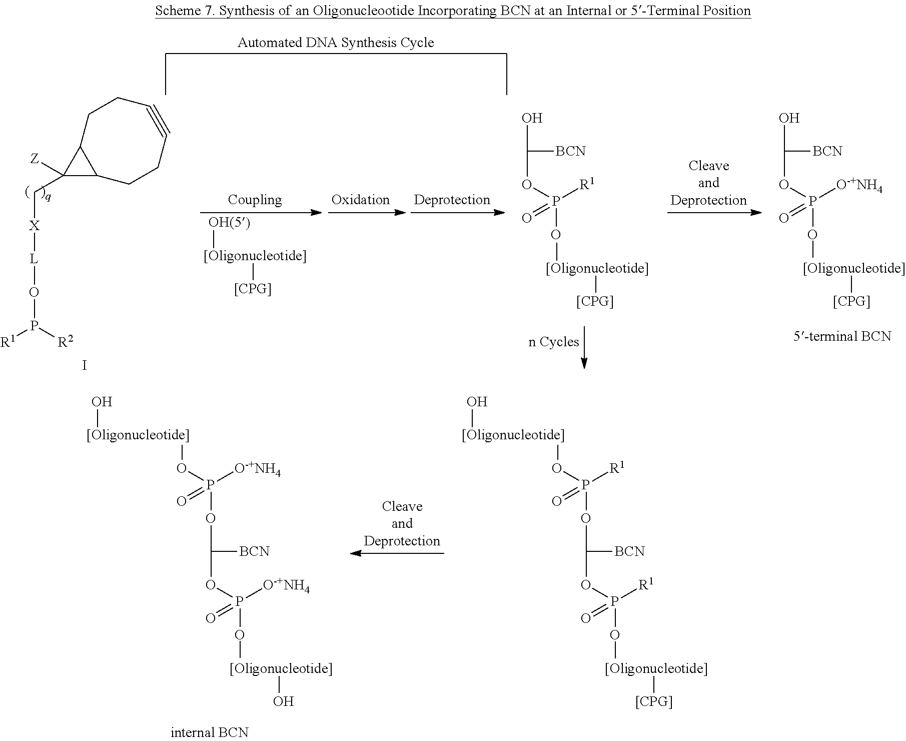

Scheme 7 illustrates a method for the introduction of a BCN-containing compound of Formula I at an internal or 5′-terminal position of an oligonucleotide. To obtain an oligonucleotide containing the BCN group at an internal position, DNA synthesis is continued after the incorporation of the compound of Formula I. To obtain an oligonucleotide containing the BCN group at the 5′-terminal position, the oligonucleotide is cleaved from the solid support after the incorporation of the compound of Formula I, and the protecting groups are removed, to afford the free oligonucleotide.

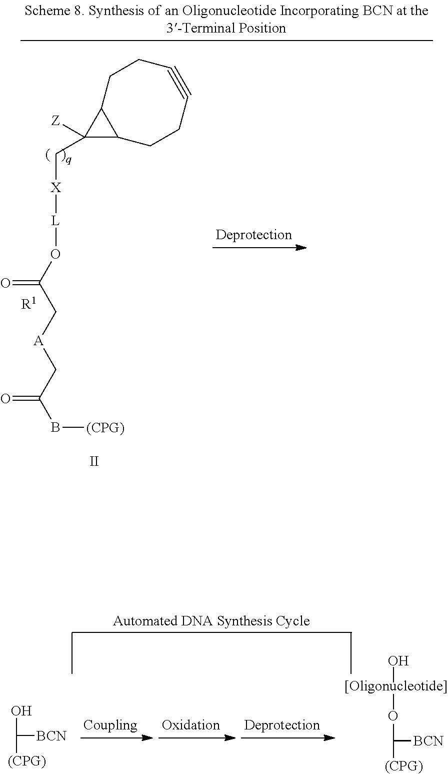

Scheme 8 illustrates a method for introduction of a BCN-containing compound of Formula II at the 3′-terminal position of an oligonucleotide. The compound of Formula II is deprotected (if necessary) to remove the DMT protecting group and reveal a free hydroxyl group. The resulting compound is employed in automated DNA synthesis to afford the oligonucleotide.

Ligation of Chemical Tags to BCN—Containing Oligonucleotides

Scheme 9 depicts proof of concept studies for the conjugation of various cofactor-azides with a 5′-BCN-containing oligonucleotide incorporating a compound of Formula I. An oligo consisting of six thymidine nucleotides is terminated with 10 using the elongation cycle of chemical reactions described above (Scheme 4) to afford 5% (10)-(T₆)-Icaa-CPG. This is achieved using the solid supported synthesis method in an automated oligonucleotide synthesizer. The oligo is cleaved from the solid support and cyanoethyl protecting groups are removed from the phosphate groups to afford 5′-(10)-(T₆). These operations are achieved through the use of methods that are well known to those skilled in the art of oligonucleotide synthesis.

The resulting 5′-BCN-containing oligonucleotide (5′-(10)-(T₆)) is dissolved in a mixture of an aqueous buffer and a water-miscible organic solvent such as acetonitrile. A solution of the cofactor-azide is then added, and the mixture is allowed to stand at room temperature. The Cu-free click reaction proceeds cleanly, affording the cofactor-oligo conjugate. The progress of the conjugation reaction can be followed by HPLC and the identity of the conjugate can be confirmed by mass spectrometry. The flexibility and robustness of this approach is shown by the preparation of 37, 38, and 39, in which diverse chemical tags have been incorporated using the catalyst-free click reaction.

Other compounds of Formula I, such as 11, 22, 23, 24, 25, 48, 49, 50, 51, and 54, may be employed under analogous conditions to prepare other 5′-BCN-containing oligonucleotides. Yet other compounds of Formula I, such as 7a, 7b, 13, 15, 19, 21, 42, 43, and 47, may also be employed to prepare BCN-containing oligonucleotides bearing BCN groups at internal positions (e.g. (T₅)-(I)-(T₅) or A-T-G-C-C-G-T-A-(I)-T-A-G-C). Finally, compounds of Formula II, such as 33a, 33b, 34a, 34b, 34c, 34d, 35, 36, 44, 45, 46, and 52, can be employed to prepare 3′-BCN-containing oligonucleotides. Regardless of the structure or position of the BCN group in the BCN-containing oligonucleotide, the Cu-free click reactions may be carried out as described above to attach a variety of chemical tags to the oligonucleotides. Further details pertaining to the use of compounds of the invention in oligonucleotide tagging are provided in the Examples.

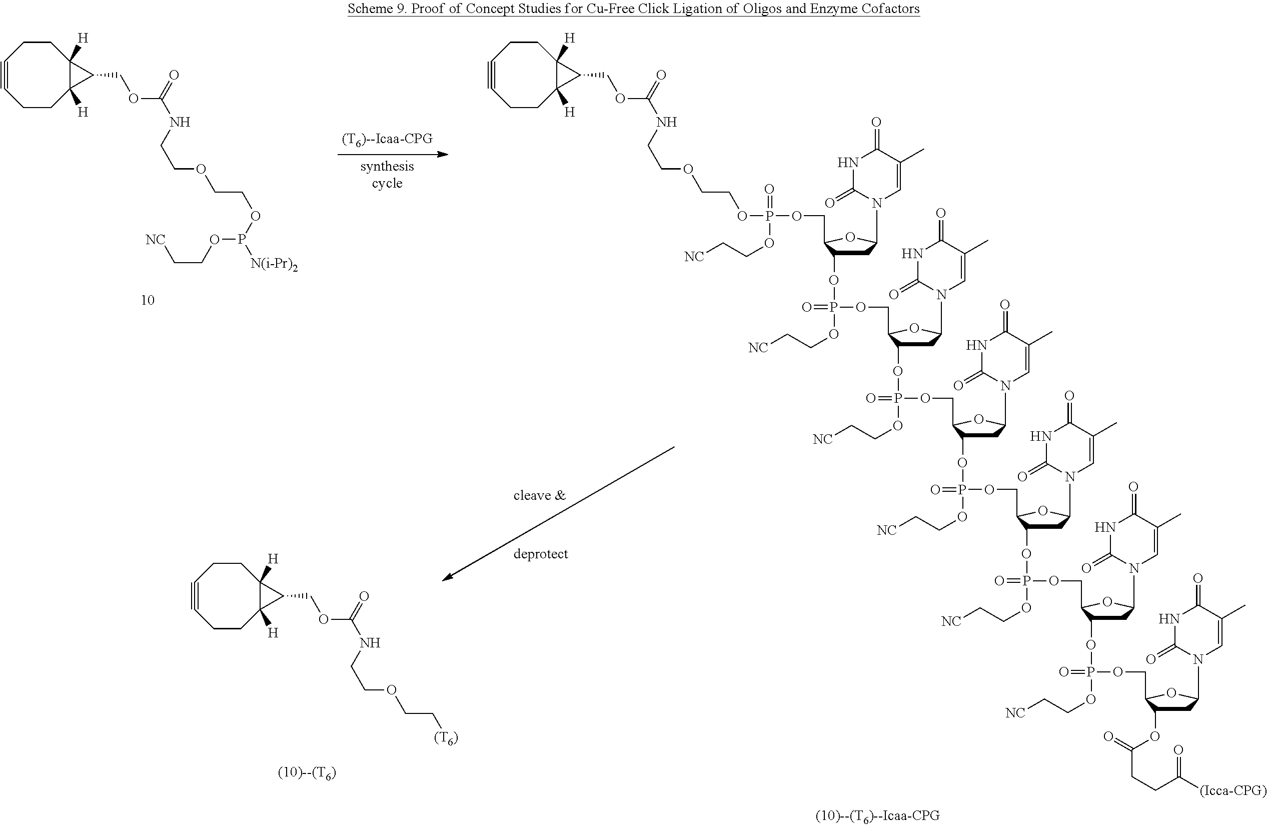

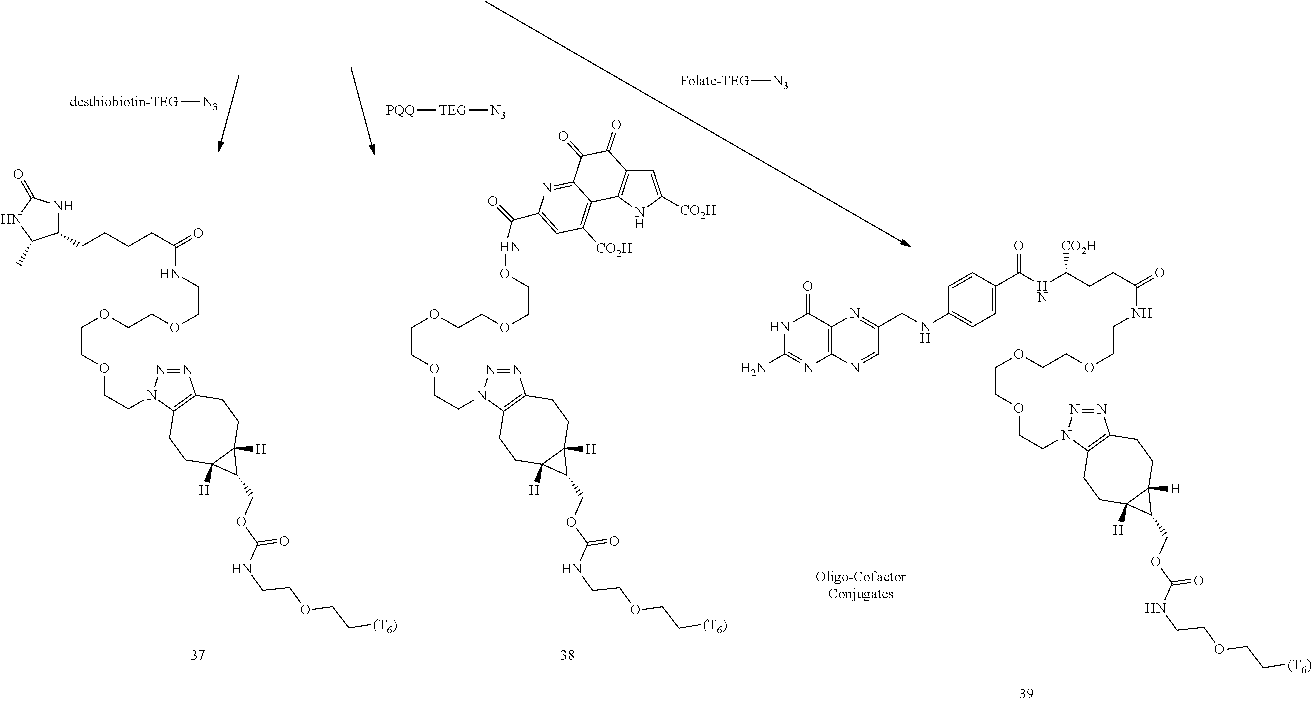

### EXAMPLES

### Example 1

### endo-Bicyclo[6.1.0]non-4-yn-9-ylmethyl (2-(2-(((2-cyanoethoxy)(diisopropylamino)-phosphino)oxy)ethoxy)ethyl)carbamate, 10

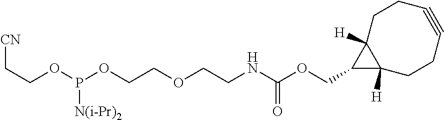

A solution of endo-bicyclo[6.1.0]non-4-yn-9-ylmethyl (2-(2-hydroxyethoxy)ethyl)carbamate(hereinafter 9) (0.42 g, 1.5 mmol) (obtained from SynAffix, B.V, Catalog No. SX-A1012) in anhydrous dichloromethane (7 mL) is treated with 3-((bis(diisopropylamino)phosphino)-oxy)propanenitrile (0.55 mL, 1.7 mmol). The resulting solution is treated with a dichloromethane solution containing 0.25M trifluoroacetic acid and 0.5M N-methylmorpholine (3.0 mL, 0.75 mmole H⁺). The reaction is stirred at room temperature for 90 minutes. After dilution with dichloromethane (20 mL), the reaction solution is washed with water (2×25 mL) and then washed with 5% aqueous NaHCO₃ (1×25 mL). The organic layer is dried over Na₂SO₄, filtered, and evaporated at reduced pressure. A slurry of Silica gel (5 g) in hexane-acetone-triethylamine (85:10:5) is packed into a 1.5 cm diameter column. The crude reaction product is dissolved in hexane-acetone-dichloromethane (80:10:10) and loaded onto the silica column. Elution with hexane-acetone-dichloromethane (80:10:10 followed by 70:20:10) and collection of 5 mL fractions allows the separation of 10 from other impurities. Pure fractions are evaporated to afford a colorless liquid (0.6 g). MS (AP+): 482 (M+H); 504 (M+Na) is consistent with the desired phosphoramidite, 10.

### Example 2

### 2-(exo-Bicyclo[6.1.0]non-4-yn-9-yl)ethyl (2-cyanoethyl) diisopropylphosphoramidite, 11

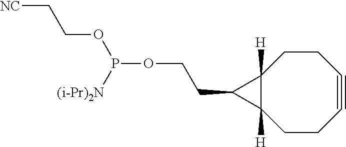

A solution of exo-BCN-ethanol (vi(c) from Example 23, 2.0 g, 12.2 mmol) is treated with 3-((bis(diisopropylamino)phosphino)-oxy)propanenitrile (4.53 mL, 14.0 mmol) according to the method of Example 1 to afford 11, as a colorless liquid. MS(AP+): 365 (M+H); 387 (M+Na) is consistent with the desired phosphoramidite, 11.

### Example 3

### endo-Bicyclo[6.1.0]non-4-yn-9-ylmethyl ((E)-3-(5′-β-dimethoxytrityl-2′-deoxyuridin-5-yl)allyl)carbamate, 12

A solution of 5-((E)-3-aminoprop-1-en-1-yl)-5′-O-dimethoxytrityl-2′-deoxyuridine (1.17 g, 2.0 mmol) (prepared according to the method of Santoro, et al., J. Am. Chem. Soc. 2000, 122(11), 2433-9) in DCM (10 mL) is treated with a solution of endo-bicyclo[6.1.0]non-4-yn-9-ylmethyl (2,5-dioxopyrrolidin-1-yl) carbonate (VIIa) (0.64 g, 2.2 mmol) (obtained from SynAffix, Catalog No. SX-A1028) in dichloromethane (10 mL). After stirring overnight at room temperature, the reaction mixture is washed with saturated aqueous NaHCO₃ and the organic layer is dried over Na₂SO₄. After filtration, the resulting solution is concentrated at reduced pressure. The residue is purified by chromatography on silica gel, eluting with a gradient of 1-5% methanol in dichloromethane. Fractions containing pure 12 are combined and concentrated at reduced pressure. The residue is dissolved in dichloromethane (50 mL) and concentrated again. Drying overnight under vacuum affords 12 as a crisp, colorless foam. MS (AP+): 762 (M+H).

### Example 4

### endo-Bicyclo[6.1.0]non-4-yn-9-ylmethyl((E)-3-(3′-O-(((2-cyanoethoxy)-diisopropylamino)phosphityl)-5′-O-dimethoxytrityl-2′-deoxyuridin-5-yl)allyl)carbamate, 13

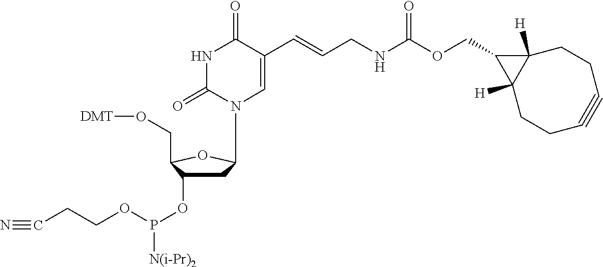

12 (from Example 3) (1.14 g, 1.5 mmol) is dissolved in anhydrous dichloromethane (10 mL). The resulting solution is treated with 3-((bis(diisopropylamino)phosphino)-oxy)propanenitrile (0.55 mL, 1.72 mmol), followed by addition of with a dichloromethane solution containing 0.25M trifluoroacetic acid and 0.5M N-methylmorpholine (3.0 mL, 0.75 mmole H⁺). The reaction solution is stirred for 4 hours at room temperature. After dilution with dichloromethane (25 mL), the reaction solution is washed with water (2×25 mL) and then washed with 5% aqueous NaHCO₃ (1×25 mL). The organic layer is dried over Na₂SO₄, filtered, and evaporated at reduced pressure. The residue is dissolved in dichloromethane (3 mL), diluted with n-pentane (3 mL) and the resulting solution is added dropwise to vigorously stirred n-pentane. The resulting suspension is allowed to stand for 15 minutes then the hazy liquor is decanted from the gummy precipitate that adheres to the flask walls. The gummy precipitate is dissolved in acetonitrile (20 mL). The solution is dried over Na₂SO₄, filtered, and concentrated at reduced pressure. The residue is dissolved in anhydrous dichloromethane (30 mL) and concentrated at reduced pressure. Further drying under vacuum overnight gives a 13 as a crisp, colorless foam. MS (AP+): 962 (M+H).

### Example 5

### endo-Bicyclo[6.1.0]non-4-yn-9-ylmethyl(6-((E)-3-(3′-O-(((2-cyanoethoxy)-diisopropylamino)phosphityl)-5′-O-dimethoxytrityl-2′-deoxyuridin-5-yl)acrylamido)hexyl)carbamate, 15

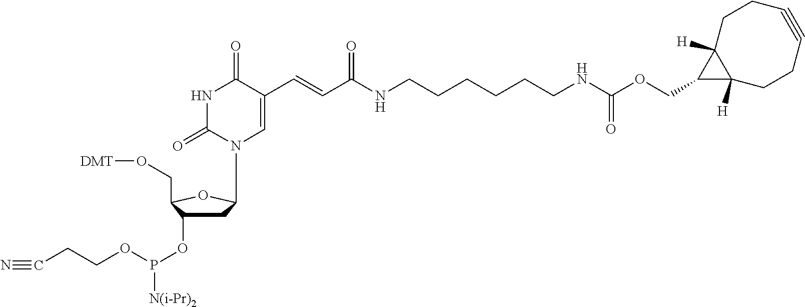

A solution of 5-[N-(6-Aminohexyl)-3-(E)-acrylamido]-5′-β-(dimethoxytrityl)-2′-deoxyuridine (obtained from Berry and Associates, Inc., Catalog No. PY 7050) is treated with VIIa according to the method of Example 3 to provide endo-Bicyclo[6.1.0]non-4-yn-9-ylmethyl(6-((E)-3-(5′-O-dimethoxytrityl-2′-deoxyuridin-5-yl)acrylamido)hexyl)carbamate (hereinafter 14), which is then phosphitylated according to the method of Example 4 to afford 15. MS (AP+): 1075, (M+H).

### Example 6

### (RS)-endo-Bicyclo[6.1.0]non-4-yn-9-ylmethyl (8-dimethoxytrityloxy-6-hydroxy-4,8-dioxa-octyl)carbamate, 6a

(R,S)-3-(3-aminopropoxy)propane-1,2-diol (1.64 g, 11 mmol) (Prepared according to the method of Misiura and Gait, WO 9117169 A1) and VIIa (2.91 g, 10 mmol) are dissolved in anhydrous tetrahydrofuran (50 mL) and stirred overnight at room temperature. The reaction solution is concentrated at reduced pressure and the residue is partitioned between ethyl acetate (50 mL) and 5% aqueous Na₂CO₃ (20 mL). The organic layer is dried over Na₂SO₄, filtered, and concentrated at reduced pressure. The residue is dissolved in anhydrous pyridine (30 mL) and treated with dimethoxytrityl chloride (3.0 g, 9.0 mmol) and the resulting solution is stirred overnight at room temperature. The reaction is concentrated at reduced pressure and the resulting residue is partitioned between ethyl acetate and saturated aqueous NaHCO₃. The organic layer is washed with saturated aqueous NaCl, dried over Na₂SO₄, filtered, and concentrated at reduced pressure. Flash chromatography on silica gel, eluting with a gradient of 5% to 45% ethyl acetate in hexanes affords 6a as a sticky foam upon evaporation of solvents at reduced pressure. MS (AP+): 628, (M+H).

### Example 7

### (RS)-endo-Bicyclo[6.1.0]non-4-yn-9-ylmethyl (8-dimethoxytrityl-6-(((2-cyanoethoxy)-diisopropylamino)phosphino)-4,8-dioxa-octyl)carbamate, 7a

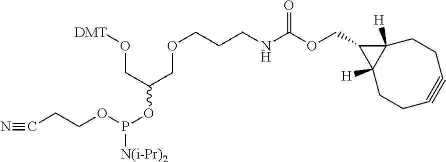

6a from Example 6 is phosphitylated according to the method of Example 1 to provide the phosphoramidite, 7a. MS (AP+): 828 (M+H).

### Example 8

### 1-(endo-Bicyclo[6.1.0]non-4-yn-9-yl)-10-(dimethoxytrityloxymethyl)-3,12-dioxo-2,8,11-trioxa-4-azapentadecan-15-oic acid on Icaa-CPG, 33a

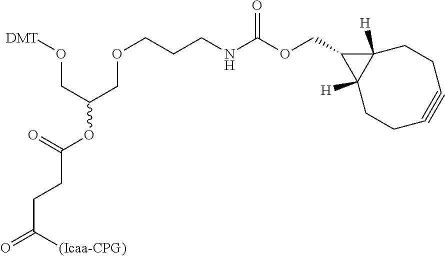

6a from Example 6 (630 mg, 1.0 mmol) is dissolved in anhydrous pyridine (5 mL) and treated with succinic anhydride (120 mg, 1.2 mmol). The resulting solution is stirred overnight at room temperature then quenched by addition of water (0.5 mL). The solution is concentrated at reduced pressure and partitioned between ethyl acetate and water (25 mL each). The organic layer is dried over Na₂SO₄, filtered, and concentrated at reduced pressure. The resulting hemisuccinate ester of 6a is dissolved in dichloromethane, treated with 1 molar equivalent of PYBOP, 2 molar equivalents of diisopropylethylamine, and 0.5 equivalents of lcaa-CPG. The resulting suspension is shaken for 16 hours at room temperature then the solid is collected by filtration. The solid is successively washed with methanol, dichloromethane, methanol, dichloromethane, acetonitrile, and dichloromethane and then dried under a flow of anhydrous nitrogen gas. Final drying of 33a is accomplished by placing in an evacuated dessicator over anhydrous CaCl₂, overnight at room temperature.

### Example 9

### 5-((E)-3-(((endo-Bicyclo[6.1.0]non-4-yn-9-ylmethoxy)carbonyl)amino)prop-1-en-1-yl)-5′-O-dimethoxytrityl-2-deoxyuridine)-3′-O-hemisuccinate on lcaa-CPG, 34a

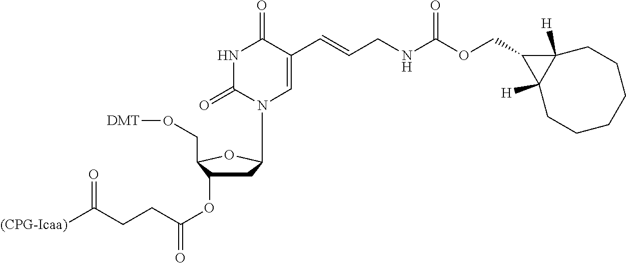

12 from Example 3 is converted to 34a according to the method of Example 8.

### Example 10

### (1R,8S)-Diethyl bicyclo[6.1.0]non-4-ene-9,9-dicarboxylate (ii(b))

To a solution of 1,5-cyclooctadiene (5.27 mL, 43.0 mmol) and Rh₂(OAc)₄ (100 mg, 0.23 mmol) in CH₂Cl₂ (5 mL) is added dropwise over 3 h a solution of diethyl diazomalonate (1.0 g, 5.37 mmol) in CH₂Cl₂ (5 mL). This solution is stirred for 24 h at room temperature. The CH₂Cl₂ is evaporated and the excess of cyclooctadiene is removed by filtration over a glass filter filled with silica (eluent: heptane). The filtrate is concentrated in vacuo and the residue is purified by column chromatography on silica gel (ethyl acetate:heptane, 1:10) to afford ii(b) (1.03 g, 72%). ¹H NMR (CDCl₃, 400 MHz): 5.65-5.57 (m, 2H), 4.10 (2×q, J=7.2 Hz, 4H), 2.41-2.29 (m, 2H), 2.15-2.06 (m, 3H), 1.83-1.70 (m, 3H), 1.31-1.23 (2×t, J=7.2 Hz, 6H).

### Example 11

### (1R,8S)-Bicyclo[6.1.0]non-4-ene-9,9-diyldimethanol (iii(b))

To a suspension of LiAlH₄ (103 mg, 2.70 mmol) in diethyl ether (10 mL) is added dropwise at 0° C. a solution of ii(b) from Example 10 (400 mg, 1.50 mmol) in diethyl ether (10 mL). Water is added carefully until the grey solid turns white. Na₂SO₄ (2 g) is added and the solid is filtered-off and washed thoroughly with diethyl ether (100 mL). The filtrate is concentrated in vacuo. The residue is purified by column chromatography on silica gel (ethyl acetate:heptane, 3:1) to afford iii(b) as a white solid (190 mg, 69%). ¹H NMR (CDCl₃, 400 MHz): 400 MHz): solid (190 mg, 69% J=4.8 Hz, 2H), 3.58 (d, J=4.8 Hz, 2H), 2.43-2.35 (m, 2H), 2.20-1.99 (m, 6H), 1.71-1.57 (m, 2H), 0.95-0.88 (m, 2H).

### Example 12

### ((1R,8S)-4,5-Dibromobicyclo[6.1.0]nonane-9,9-diyl)dimethanol (iv(b))

The diol, iii(b) from Example 11, (145 mg, 0.796 mmol) is dissolved in CH₂Cl₂ (5 mL). At 0° C. a solution of Br₂ (45 (45 L). Ammol) in CH₂Cl₂ (1 mL) is added dropwise until the yellow color persists. The reaction mixture is quenched with a 10% Na₂S₂O₃ solution (5 mL) and extracted with CH₂Cl₂ (2×20 mL). The organic layer is dried (Na₂SO₄) and concentrated in vacuo. The residue is purified by column chromatography on silica gel (EtOAc:heptane, 5:1) afford iv(b) (235 mg, 86%) as a white solid. ¹H NMR (CDCl₃, 400 MHz): 4.87-4.78 (m, 2H), 3.96-3.88 (m, 2H), 3.60 (d, J=5.2 Hz, 2H), 2.75-2.63 (m, 2H), 2.32-2.22 (m, 3H), 2.20-2.13 (m, 1H), 2.05-1.94 (m, 2H), 1.74-1.57 (m, 2H), 1.13-0.99 (m, 2H).

### Example 13

BCN-dimethanol, v(b). To a solution of the dibromide, iv(b) from Example 12, (100 mg, 0.292 mmol) in THF (5 mL) is added dropwise at 0° C. a solution of KOtBu (1.29 mL, 1 M in THF, 1.29 mmol). The solution is then refluxed for 1.5 h. After cooling to room temperature, the mixture is quenched with saturated NH₄Cl-solution (20 mL), and extracted with CH₂Cl₂ (3×20 mL). The organic layer is dried (Na₂SO₄) and concentrated in vacuo. The residue is purified by column chromatography on silica gel (ethyl acetate) to afford v(b) (24 mg, 46%) as a white solid. ¹H NMR (CDCl₃, 400 MHz): 3.89 (bs, 2H), 3.63 (bs, 2H), 2.58 (bs, 2H), 2.34-2.20 (m, 6H), 1.68-1.59 (m, 2H), 0.89-0.82 (m, 2H).

### Example 14

### (9-((dimethoxytrityloxy)methyl)bicyclo[6.1.0]non-4-yn-9-yl)methanol (vi(b))

A solution of v(b) from Example 13 (1.8 g, 10 mmol) is dissolved in anhydrous pyridine (50 mL) and treated with small portions of DMT-Cl (10×339 mg, 10 mmol) at 20 minute intervals. The resulting solution is stirred for an additional 3 hours. After concentration at reduced pressure, the residue is partitioned between ethyl acetate (100 mL) and saturated aqueous NaHCO₃ (50 mL). The organic layer is washed with saturated aqueous NaCl (50 mL), dried over Na₂SO₄, filtered, and concentrated at reduced pressure. The residue is purified by chromatography on silica gel, eluting with a gradient of 10-60% ethylacetate in hexanes to afford vi(b) upon evaporation of solvents. MS (AP+): 483 (M+H).

### Example 15

### 2-(endo-bicyclo[6.1.0]non-4-yn-9-yl)ethyl (3-(3-(bis(4-methoxyphenyl)(phenyl)methoxy)-2-(((2-cyanoethoxy)(diisopropylamino)phosphino)oxy)propoxy)propyl)carbamate (42)

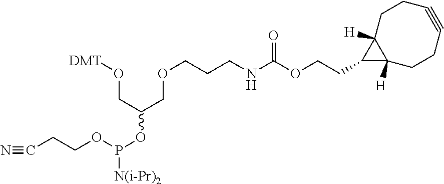

(RS) 2-(endo-Bicyclo[6.1.0]non-4-yn-9-yl)ethyl (3-(3-(bis(4-methoxyphenyl)(phenyl)methoxy)-2-hydroxypropoxy)propyl)carbamate (41) is first prepared by substituting 40e, from Example 25 for VIIa in the method of Example 6. Purified 41 is then phosphitylated according to the method of Example 1 to provide the phosphoramidite, 42. MS (AP+): 843 (M+H).

### Example 16

### endo-Bicyclo[6.1.0]non-4-yn-9-yl-formaldehyde (17)

To a cooled (0° C.) suspension of BCN-methanol (180 mg, 1.20 mmol) in CH₂Cl₂ (10 mL) is added Dess-Martin periodinane (0.68 g, 1.6 mmol) and the suspension is stirred for 4 h at rt. After this time, water (10 mL) is added, the solvent layers are separated and the CH₂Cl₂ layer is dried on MgSO₄ and filtered. The filtrate is concentrated in vacuo at 0° C. and the residue is purified by column chromatography on silica gel (CH₂Cl₂) to afford 17 as a white solid (53 mg, 30%). ¹H NMR (CDCl₃, 300 MHz): 9.6 (d, 1H), 2.33-2.22 (m, 9H), 1.57-1.54 (m, 3H).

### Example 17

### endo-Bicyclo[6.1.0]non-4-yn-9-yl-methylamine (18)

To a stirred solution of endo-bicyclo[6.1.0]non-4-yn-9-ylformaldehyde (15 mg, 0.10 mmol) in MeOH (5 mL) is added NH₄OAc (0.50 g) and NaCNBH₃ (7.5 mg, 0.12 mmol). After stirring overnight, the mixture H₂O (10 mL) is added and the resulting solution is concentrated in vacuo. Diethyl ether (10 mL) and saturated Na₂CO₃ (1 mL) are added, the mixture shaken and the layers are allowed to separate. The aqueous phase is extracted with CH₂Cl₂ (2×10 mL). The combined organic layers are dried (MgSO₄) and filtered. The filtrate is concentrated in vacuo, applied onto a column of silica gel and eluted with a mixture of MeOH/NH₃ (7 N in MeOH)/CH₂Cl₂ (1:1:48→3:1:46→3:3:44). Pure fractions are concentrated, re-dissolved in CH₂Cl₂ and concentrated again to afford pure 18 (9 mg, 60%). ¹H NMR (CDCl₃)::MR(CDCl).at 2.26-2.16 (m, 3H), 1.65-1.50 (m, 1H), 1.40-1.08 (m, 6H), 0.90-0.82 (m, 2H).

### Example 18

### (RS)-endo-bicyclo[6.1.0]non-4-yn-9-ylmethyl-(3-((3-(((2-cyanoethoxy)(diisopropylamino)phosphino)oxy)-5-dimethoxytrityloxypentyl)oxy)propyl)carbamate (7b)

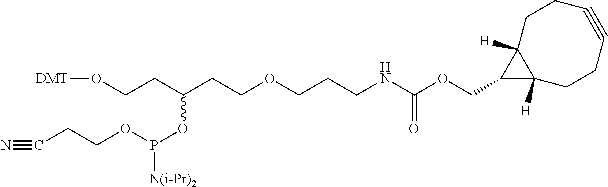

The reaction of (R,S)-5-(3-aminopropoxy)pentane-1,3-diol (synthesized from 1,3,5-pentanetriol (Obtained from Beta Pharma Scientific Products, Catalog No. 86-43517) in 4 steps according to the analogous method of Misiura and Gait, WO 9117169 A1) under the conditions of Example 6 affords (RS)-endo-bicyclo[6.1.0]non-4-yn-9-ylmethyl-(3-((3-hydroxy-5-dimethoxytrityloxypentyl)oxy)propyl)carbamate (hereinafter 6b), which is phosphitylated according to the method of Example 7 to afford 7b.

### Example 19

### 1-(endo-Bicyclo[6.1.0]non-4-yn-9-yl)-11-(dimethoxytrityloxymethyl)-3,13-dioxo-2,8,12-trioxa-4-azahexadecan-16-oic acid on Icaa-CPG, 33b

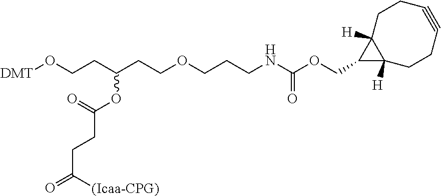

The title compound is obtained from 6b (from Example 18) according to the method of Example 8.

### Example 20

### endo-Bicyclo[6.1.0]non-4-yn-9-ylmethyl(6-((E)-3-(3′-O-hemisuccinate-5′-O-dimethoxytrityl-2′-deoxyuridin-5-yl)acrylamido)hexyl)carbamate on Icaa-CPG, 34b

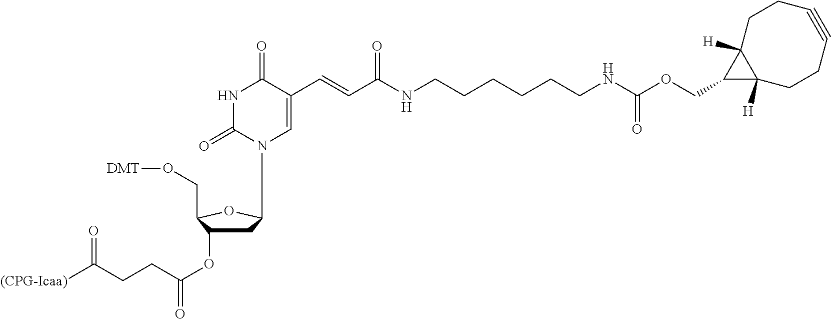

The title compound is obtained from 14 (from Example 5) according to the method of Example 9.

### Example 21

### (Z)-exo-Bicyclo[6.1.0]non-4-ene-9-carbaldehyde, exo-iii(c)

(Z)-exo-Bicyclo[6.1.0]non-4-en-9-ylmethanol (exo-iii(a)) (prepared according to Dommerholt et al., Angewande Chemie, International Edition, 2010, 49, 9422-9425) (5.2 g, 26.6 mmol) is dissolved in dichloromethane (300 mL). Pyridinium chlorochromate (10.5 g, 48.5 mmol) is added. The resulting reaction mixture is stirred for 2 hours and subsequently filtered over a short path of silicagel. The filtrate is concentrated and purified by column chromatography (dichloromethane), yielding 4.9 g of the aldehyde, exo-iii(c). This material is used without further purification in Example 22.

### Example 22

### (Z)-exo-2-(Bicyclo[6.1.0]non-4-en-9-yl)ethanol, exo-iv(c)

Under an atmosphere of argon, (methoxymethyl)triphenylphosphonium chloride (17.1 g; 50 mmol) is suspended in anhydrous THF (100 mL) and cooled to 0° C. Potassium tert-butoxide (5.6 g; 50 mmol) is added and the resulting mixture is stirred for 20 minutes. A solution of exo-iii(c) (Example 21, 4.95 g; 33.0 mmol) in anhydrous THF (100 mL) is added. The resulting reaction mixture is stirred for 15 minutes and then poured into a mixture of diethylether and water (200 mL/200 mL). The aqueous phase is separated and extracted a second time with diethylether (100 mL). The two combined organic layers are dried (Na₂SO₄) and concentrated at reduced pressure. The residue is dissolved in tetrahydrofuran (200 mL) and aqueous hydrochloric acid (1M, 100 mL) is added. The resulting mixture is heated to reflux for 45 minutes, cooled to room temperature and poured into a mixture of diethylether and water (200 mL/200 mL). The aqueous phase is separated and extracted a second time with diethylether (100 mL). The two combined organic layers are dried (Na₂SO₄) and concentrated at reduced pressure. The residue is dissolved in methanol (200 mL) and placed under an atmosphere of argon. After cooling the reaction mixture to 0° C., NaBH₄ (1.89 g; 50 mmol) is added in portions. The mixture is stirred for 15 minutes, quenched with saturated aqueous ammonium chloride (100 mL) and partitioned between diethylether (200 mL) and water (100 mL). The aqueous phase is separated and extracted with diethylether (2×200 mL). The three combined organic layers are dried (Na2SO4) and concentrated at reduced pressure. The crude product is purified by column chromatography on silica gel, eluting with a 10-25% gradient of ethylacetate in pentane to provide 4.22 g (77%) of exo-iv(c). ¹H-NMR (300 MHz, CDCl₃) δ 5.70-5.56 (m, 2H), 3.68 (t, J=6.6 Hz, 2H), 2.39-1.94 (m, 6H), 1.51 (q, J=6.7 Hz, 2H), 1.44-1.23 (m, 3H), 0.71-0.57 (m, 2H), 0.30-0.20 (m, 2H).

### Example 23

### exo-BCN-ethanol, exo-yl(c)

A solution of bromine (1.37 mL, 26.7 mmol) in dichloromethane (25 mL) is added dropwise to an ice-cold solution of exo-iv(c) (Example 22, 4.22 g, 25.4 mmol) in dichloromethane (100 mL). Subsequently, 10% aqueous Na₂S₂O₃ (50 mL) is added. The aqueous phase is separated and extracted a second time with dichloromethane (50 mL). The two combined organic layers are then dried (Na₂SO₄) and concentrated at reduce pressure to afford exo-v(c), 8.33 g (100%). Without further purification, exo-v(c) is dissolved in anhydrous THF (100 mL), placed under an argon atmosphere, and cooled to 0° C. A solution of potassium tert-butoxide (9.3 g; 83 mmol) in anhydrous THF (100 mL) is added dropwise. The resulting reaction mixture is heated to 70° C., stirred for 30 minutes, and quenched with saturated NH₄Cl_{(aq)} (100 mL). The resulting mixture is extracted twice with diethylether (200 mL). The two combined organic layers are then dried (Na₂SO₄) and concentrated at reduced pressure. The crude product is purified chromatography on silica gel to afford exo-vi(c), (2.57 g; 15.6 mmol; 62%) as a slightly yellow solid/wax. ¹H NMR (300 MHz, CDCl₃) δ 3.71 (t, J=6.5 Hz, 2H), 2.46-2.07 (m, 6H), 1.63-1.54 (m, 2H), 1.44-1.22. endo-vi(c) is likewise produced from produced from endo-iv(c). endo-iv(c), in turn, is obtained from endo-iii(a) (prepared according to Dommerholt et al., Angewande Chemie, International Edition, 2010, 49, 9422-9425) according to the procedures of Examples 21 and 22.

### Example 24

### exo-BCN-propanol, vi(d)

Starting with iv(c) from Example 22, the methods of Examples 21-23 are employed to provide vi(d).

### Example 25

### 2-(endo-bicyclo[6.1.0]non-4-yn-9-yl)ethyl (2,5-dioxopyrrolidin-1-yl) carbonate(40e)

Under ambient atmosphere, endo-vi(c) (Example 23, 83 mg; 0.51 mmol) is dissolved in acetonitrile (10 mL). N,N′-disuccinimidylcarbonate (230 mg; 0.90 mmol) is added, followed by triethylamine (213 μL; 155 mg; 1.53 mmol). The resulting mixture is stirred for 4 hours at room temperature. Ethylacetate (20 mL) is then added and the organic mixture is washed with water (3×20 mL), dried (Na₂SO₄) and concentrated. The crude is purified with column chromatography (ethylacetate/pentane 1/2), yielding 114 (0.37 mmol; 73%)mg of 40e. ¹H NMR (300 MHz, CDCl₃) δ 4.37 (t, J=6.8 Hz, 2H), 2.84 (s, 4H), 2.39-2.14 (m, 6H), 1.83-1.74 (m, 2H), 1.62-1.38 (m, 3H), 1.07-0.93 (m, 1H), 0.92-0.78 (m, 2H). 2-(exo-bicyclo[6.1.0]non-4-yn-9-yl)ethyl (2,5-dioxopyrrolidin-1-yl) carbonate(40×) is likewise produced from exo-vi(c).

### Example 26

### 3-((((exo-bicyclo[6.1.0]non-4-yn-9-yl)ethoxy)propyl)disulfanyl)propyl (2-cyanoethyl) diisopropylphosphoramidite (49)

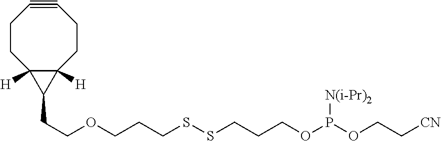

A solution of exo-vi(c) from Example 23 (1.64 g, 10 mmol) in dichloromethane (50 mL) is treated with triethylamine (1.67 mL, 12 mmol) and methanesulfonyl chloride (0.85 mL, 11 mmol), stirring overnight at room temperature. The resulting solution is washed successively with water (50 mL), 0.1M aqueous KH₂PO₄ (50 mL), and water (50 mL). The dichloromethane layer is dried over Na₂SO₄, and filtered. The resulting solution is concentrated at reduced pressure to provide 2-(exo-bicyclo[6.1.0]non-4-yn-9-yl)ethyl methanesulfonate (2.4 g). This material is dissolved in anhydrous THF (25 mL) and added to a solution formed by the mixing 3,3′-disulfanediylbis(propan-1-ol) (5.5 g, 30 mmol), potassium-t-butoxide (3.3 g, 29.5 mmol) and anhydrous THF (60 mL). The reaction is heated at reflux under a nitrogen atmosphere for 3 hours. After cooling to room temperature, water (2 mL) is added and the reaction mixture is concentrated to half its original volume at reduced pressure. The concentrate is partitioned between ethyl acetate (100 mL), hexane (100 mL) and water (100 mL). The organic layer is dried over Na₂SO₄ and filtered. The resulting solution is concentrated at reduced pressure. The crude product is purified by chromatography on silica to afford 3-((3-(2-(exo-bicyclo[6.1.0]non-4-yn-9-yl)ethoxy)propyl)disulfanyl)propan-1-ol (1.9 g). This material is treated according to the method of Example 1 to afford 49, as a viscous oil. MS(AP+): 529 (M+H), 551 (M+Na).

### Example 27

### 2-(endo-bicyclo[6.1.0]non-4-yn-9-yl)ethyl (2-(2-(((2-cyanoethoxy)(diisopropylamino)phosphino)oxy)ethoxy)ethyl)carbamate (54)

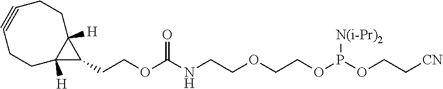

Under ambient atmosphere, 40e (112 mg; 0;.37 mmol) is dissolved in dichloromethane (5 mL). Triethylamine (154 μL; 112 mg; 1.11 mmol) and 2-(2-aminoethoxy)ethanol (56 μL; 59 mg; 0.56 mmol) are added. The mixture is stirred for 30 minutes at room temperature and then diluted with Et₂O (30 mL). The organic mixture is washed with a saturated aqueous solution of ammonium chloride (2×30 mL), washed with a saturated aqueous solution of sodium bicarbonate (2×30 mL), dried and concentrated, yielding 82 mg (0.28 mmol; 76%) mg of 2-(endo-bicyclo[6.1.0]non-4-yn-9-yl)ethyl (2-(2-hydroxyethoxy)ethyl)carbamate, 53 as a colorless syrup. Subsequently, 53 is treated according to the procedure of Example 1 to provide 54. MS (AP+): 496 (M+H); 5518 (M+Na).

### Example 28

### 5-BCN-T₆-oligo

Using a Millipore Expedite (8900 series) nucleic acid synthesis system (Billerica, Mass.), freshly prepared reagent solutions are installed in the reagent bottles as follows:
Wash A—anhydrous acetonitrile
Deblock—3% Trichloroacetic acid in anhydrous dichloromethane
Oxidizer—0.02M iodine in tetrahydrofuran/water/pyridine
Capping reagent A—acetic anhydride/anhydrous tetrahydrofuran
Capping reagent B—16% 1-methylimidazole in anhydrous tetrahydrofuran/pyridine
Wash reagent—anhydrous acetonitrile
Activator—0.25M 5-ethylthiotetrazole in anhydrous acetonitrile
Amidites: Thymidine-CEP and compound 10 from Example 1 (0.067M solutions in anhydrous acetonitrile)

The reagent lines were purged and pumps primed. Two synthesis columns containing 200 nM of DMT-T-Icaa-CPG were installed.

The instrument run parameters were then set as follows:

Column—1:
Sequence—3′-TTTTTTX-5′ (wherein T denotes a Thymidine residue and X denotes the BCN tag derived from 10.)
Protocol—CYCLE T (a 23 step protocol for reagent additions, reaction times, and washes known to be optimized for each coupling of Thymidine-CEP, as provided in the synthesizer software.)
Final DMT—On (The BCN tag is not subjected to DMT cleavage reagent since DMT protection is not present.)

Column—2:
Sequence—3′-TTTTTT-5′
Protocol—CYCLE T
Final DMT—Off
   5′-BCN-T₆-Icaa-CPG is synthesized in column 1 using CYCLE T conditions for each T residue and for the final coupling of 10. T₆-Icaa-CPG is synthesized in column 2 using CYCLE T conditions for each T residue. The output of the colorimetric monitoring of each deblock step is recorded by the synthesizer's computer. The integrated values for each of the 6 deblock steps are consistent with the successful synthesis of T₆-Icaa-CPG on both columns. In order to verify that the coupling of 10 was successful, each column is washed twice with 3 mL of 10% diethylamine in acetonitrile at room temperature, washed with 3 mL of acetonitrile, and treated with 3 mL of 28-30% ammonium hydroxide for 15 minutes at room temperature in order to remove the cyanoethyl protecting groups and cleave the oligonucleotide from the CPG support. The resulting solutions of 5′-BCN-T₆-oligo and T₆-oligo are each treated with 25 uL of triethylamine and then sparged with a stream of nitrogen until the volume was reduced to approximately 1.5 mL. The concentrated solutions are then frozen and lyophilized. Reversed phase HPLC analysis on a Waters Spherisorb ODS-2 column (150×4.6 mm) eluting at 1.0 mL/min with a 30 minute gradient of 5 to 35% acetonitrile in 0.1 M triethylammonium acetate shows a retention time for T₆-oligo of 11.3 minutes (DNA product from column 2) and a retention time for 5′-BCN-T₆-oligo of 18.1 minutes (DNA product from column 1). Furthermore, an integration ratio of 99 (5′-BCN-T₆-oligo) to 1 (T₆-oligo) is observed for the peaks in the HPLC chromatogram of DNA product from column 1, thereby confirming the successful coupling of 10 at the 5′-terminus of the oligonucleotide with high efficiency.

### Example 29

### Cu-free click conjugation of Desthiobiotin-TEG-Azide and 5′-BCN-T₆-oligo to provide 37

A solution composed of 5′-BCN-T₆-oligo (−90 nmol), desthiobiotin-TEG-azide (Berry & Associates, Inc. catalog no. BT 1075, 2400 nMol), 0.1M aqueous triethylammonium acetate (pH 7, 0.75 mL) and acetonitrile (0.15 mL) is allowed to stand at room temperature. The progress of the Cu-free click reaction is monitored by HPLC using the HPLC method described in Example 10. A new peak with a retention time of 18.6 minutes appears and the peak corresponding to 5′-BCN-T₆-oligo at 18.1 minutes disappears. The reaction is complete in 75 minutes. The resulting solution of 37 is frozen and lyophilized.

Calculated molecular weight: 2,521.0

Observed by mass spectrometry: 2,521.2

### Example 30

### Cu-free click conjugation of Methoxatin-TEG-Azide and 5′-BCN-T₆-oligo to provide 38

A solution composed of 5′-BCN-T₆-oligo (˜90 nmol), Methoxatin-TEG-azide (Berry & Associates, Inc. 180 nMol), 0.1M aqueous triethylammonium acetate (pH 7, 0.45 mL) and acetonitrile (0.05 mL) is allowed to stand at room temperature. The progress of the Cu-free click reaction is monitored by HPLC using the HPLC method described in Example 16. A new peak with a retention time of 14.9 minutes appears and the peak corresponding to 5′-BCN-T₆-oligo at 18.1 minutes disappears. The reaction is complete in 36 hours. The resulting solution of 38 is frozen and lyophilized.

Calculated molecular weight: 2,653.0

Observed by mass spectrometry: 2,653.4

### Example 31

### Cu-free click conjugation of Folate-TEG-Azide and 5′-BCN-T₆-oligo to provide 39

A solution composed of 5′-BCN-T₆-oligo (˜90 nmol), Folate-TEG-azide (Berry & Associates, Inc. catalog no. FC 8150, 180 nMol), 0.1M aqueous triethylammonium acetate (pH 7, 0.45 mL) and acetonitrile (0.05 mL) is allowed to stand at room temperature. The progress of the Cu-free click reaction is monitored by HPLC using the HPLC method described in Example 10. A new peak with a retention time of 15.9 minutes appears and the peak corresponding to 5′-BCN-T₆-oligo at 18.1 minutes disappears. The reaction is complete in 12 hours. The resulting solution of 39 is frozen and lyophilized.

Calculated molecular weight: 2,748.1

Observed by mass spectrometry: 2,747.5

### Example 32

### 5′-BCN-T₆-oligo with a short spacer. Substitution of 11 (example 2) for 10 in the method of Example 26 provides the analogous 5′-BCN-T₆-oligo with a shorter spacer between the BCN tag and the oligo

Calculated molecular weight: 1988.4

Observed by mass spectrometry: 1988.6

What is claimed is:

1. A compound of Formula I: 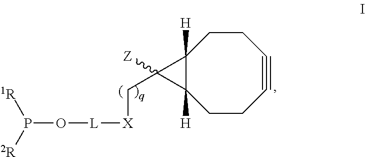
wherein:
q is 1, 2, or 3;
R¹— and R²— are independently N≡CCH₂CH₂O—, (C₁-C₆ alkyl)O—, or (C₁-C₆ alkyl)₂N—;
Z— is H—, or DMT-OCH₂—;
—X— and -L- are either both absent or both present;
—X— is absent or is —O—, —NH—, —S—, —NHCO₂—, —O₂CNH—, —NHCONH—, —NHCSNH—, or —CONH—; and
-L- is absent or is selected from a group consisting of —(CH₂)ₙ—, —(CH₂CH₂O)ₙ(CH₂)ₘ—, —(CH₂CH₂CH₂O)ₙ(CH₂)ₘ—, —(CH₂)₃S₂(CH₂)₃—, —(CH₂)₆S₂(CH₂)₆—, —(CH₂)₂O(CH₂)₃S₂(CH₂)₃O(CH₂)₂—, —CH(CH₂O-DMT)CH₂—, —CH(CH₂O-DMT)CH₂O(CH₂)ₘ—, —CH(CH₂CH₂O-DMT)CH₂CH₂—, —CH(CH₂CH₂O-DMT)CH₂CH₂O(CH₂)ₘ—, 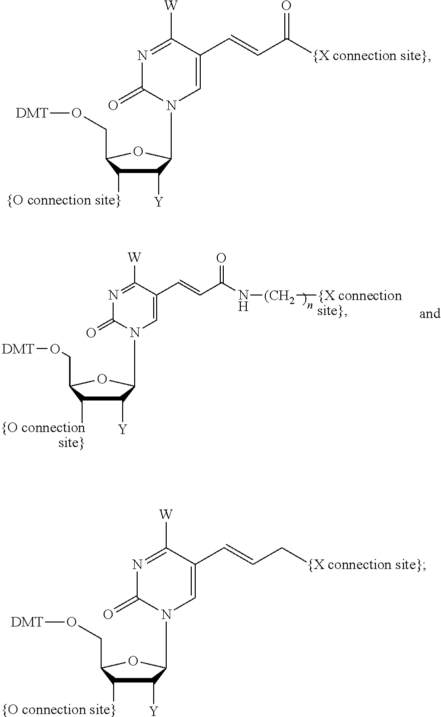
wherein n is 2-6, m is 2-3, Y is H, O-TBS, O-POM, or O-TOM, and W is OH, N═CHN(CH₃)₂, NHCOPh, or NHCOCH₃.

2. A compound according to claim 1, wherein R¹— is N≡CCH₂CH₂O— and R²— is (i-Pr)₂N.

3. A compound according to claim 1, wherein Z— is H— and —X— is —O—, —NH—, or —NHCO₂— or is absent.

4. A compound according to claim 1, wherein -L- is —(CH₂)ₙ—, —(CH₂CH₂O)ₙ(CH₂)ₘ—, or —(CH₂CH₂CH₂O)ₙ(CH₂)ₘ— or is absent.

5. A compound according to claim 1, wherein -L- is —(CH₂)₃S₂(CH₂)₃—, —(CH₂)₆S₂(CH₂)₆—, or —(CH₂)₂O(CH₂)₃S₂(CH₂)₃O(CH₂)₂—.

6. A compound according to claim 1, wherein -L- is

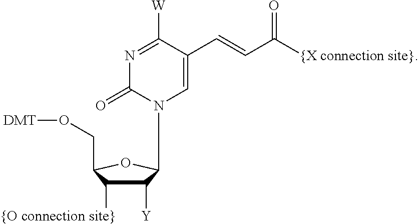

7. A compound according to claim 1, wherein -L- is

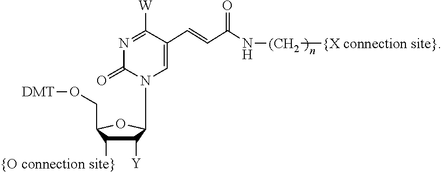

8. A compound according to claim 1, wherein -L- is

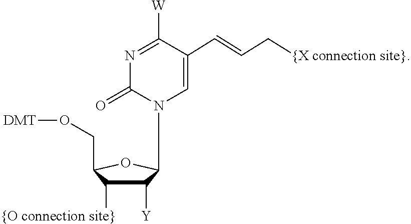

9. A compound according to claim 1, wherein —X— and -L- are both present when q is 1.

10. A compound according to claim 1, wherein q is 1.

11. A compound according to claim 1, wherein q is 2.

12. A compound according to claim 1, wherein q is 3.

13. A compound according to claim 1, wherein the compound is selected from a group consisting of:

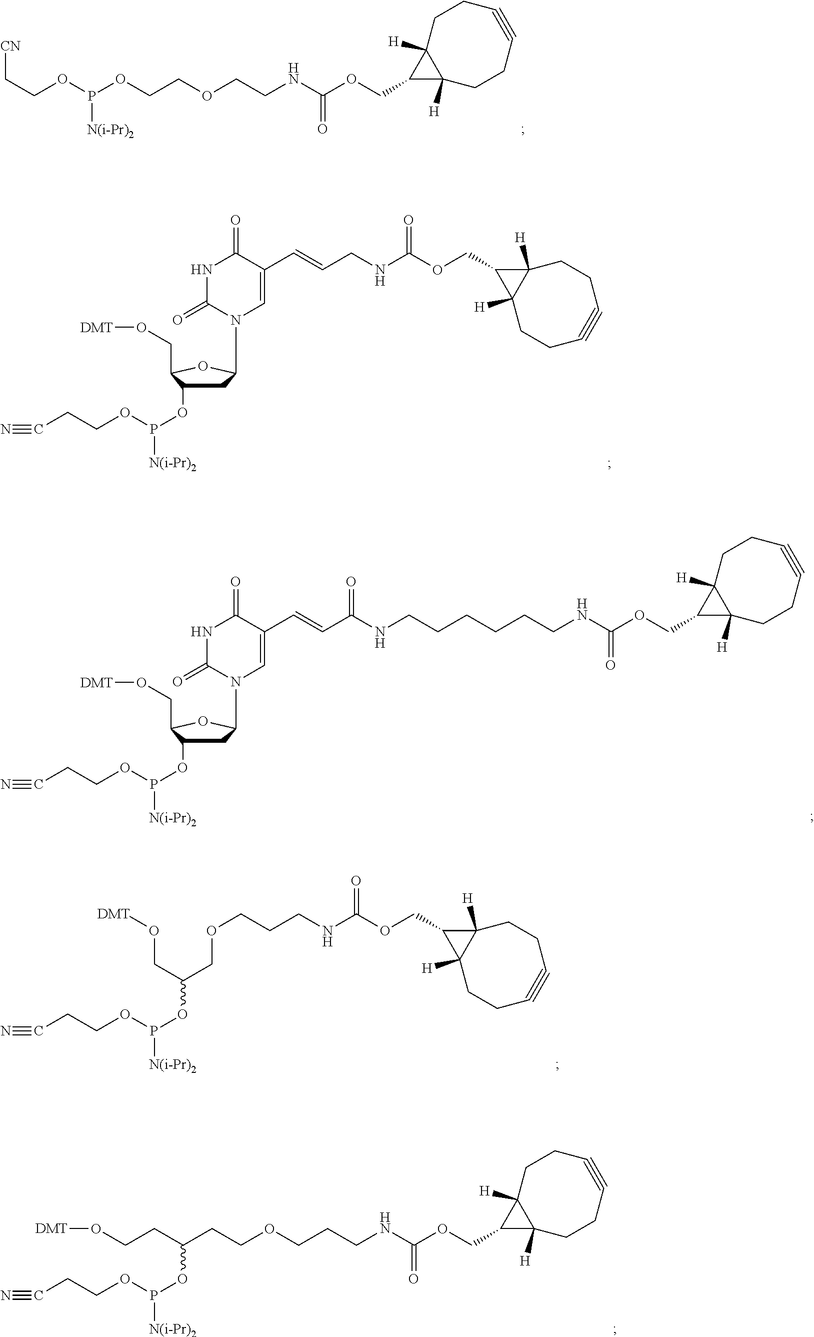

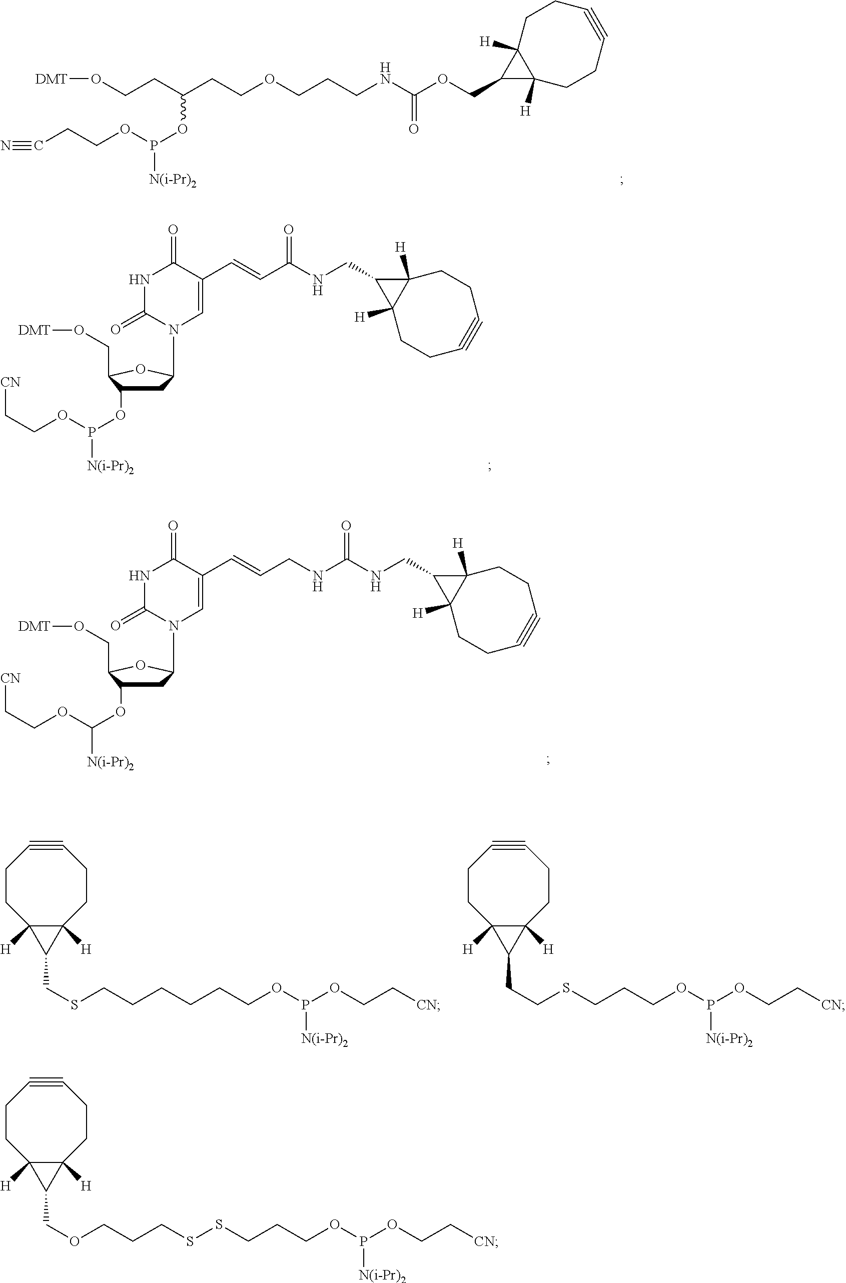

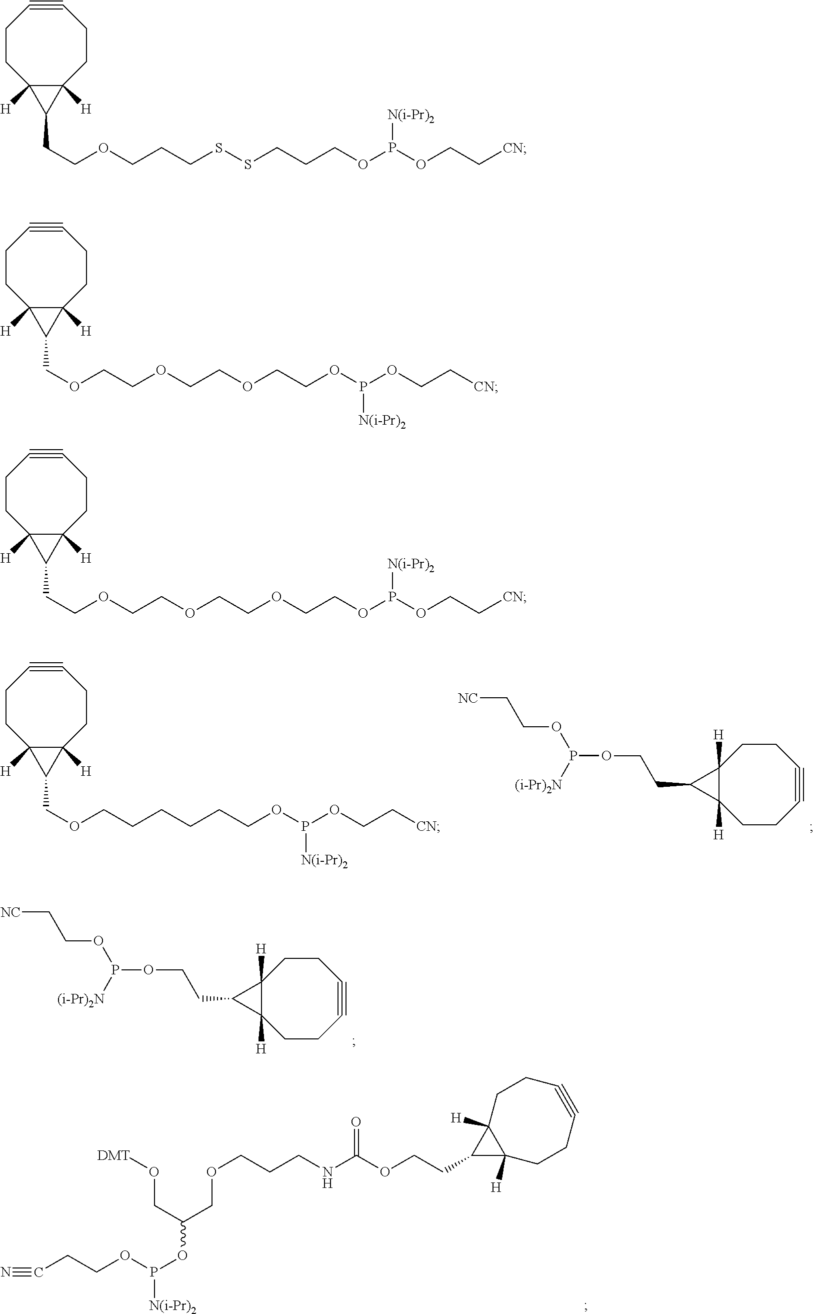

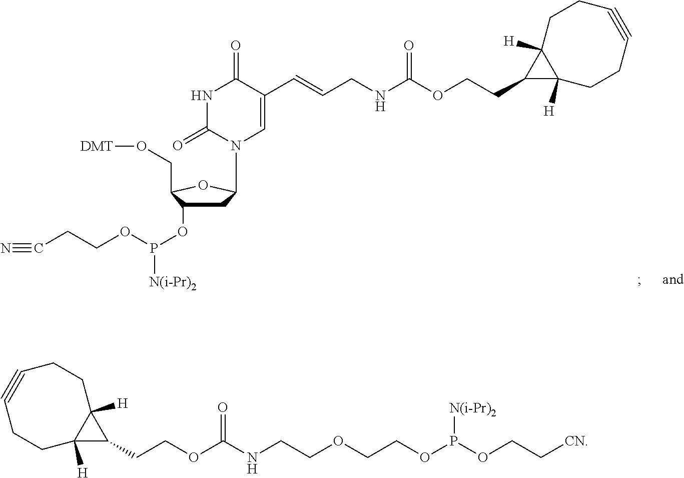

14. The compound according to claim 1 that is

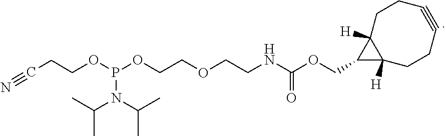 [SMILES: CC(C)N(C(C)C)P(OCCC#N)OCCOCCNC(=O)OC[C@@H]1[C@@H]2CCC#CCC[C@@H]21]

15. The compound according to claim 1 that is

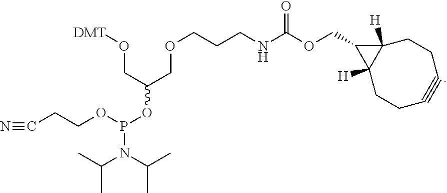

16. The compound according to claim 1 that is

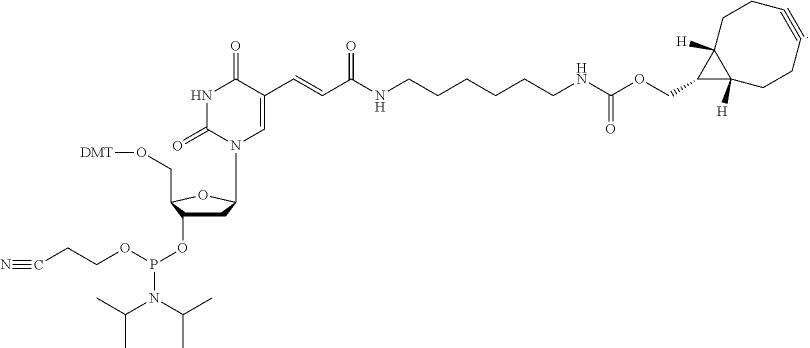

17. The compound according to claim 1 that is

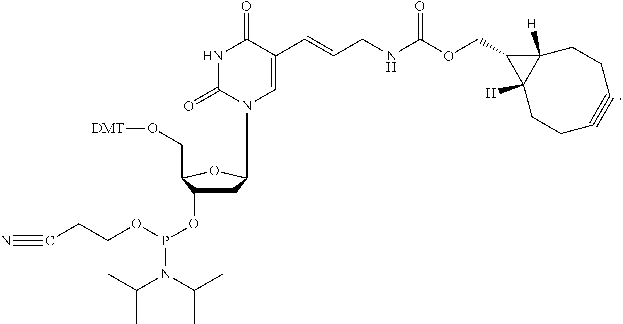

18. The compound according to claim 1 that is

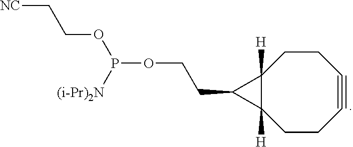

19. The compound according to claim 1 that is

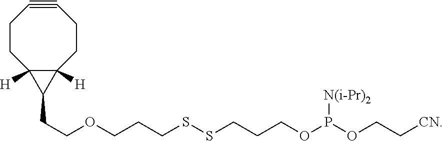

20. A compound of Formula II: 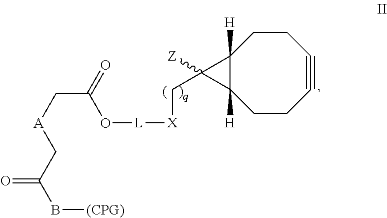
wherein:
q is 1, 2, or 3;
-A- is absent or is —O— or —O—(C₆H₄)—O—;
—B— is Icaa or aminopropyl;
Z— is H—, DMT-OCH₂—, or HOCH₂—;
—X— and -L- are either both absent or both present;
—X— is absent or is —O—, —NH—, —S—, —NHCO₂—, —O₂CNH—, —NHCONH—, —NHCSNH—, or —CONH—; and
-L- is absent or is selected from a group consisting of —CH(CH₂O-DMT)CH₂—, —CH(CH₂O-DMT)CH₂O(CH₂)ₘ—, —CH(CH₂CH₂O-DMT)CH₂CH₂—, —CH(CH₂CH₂O-DMT)CH₂CH₂O(CH₂)ₘ—, —CH(CH₂OH)CH₂—, —CH(CH₂OH)CH₂O(CH₂)ₘ—, —CH(CH₂CH₂OH)CH₂CH₂—, —CH(CH₂CH₂OH)CH₂CH₂O(CH₂)ₘ—, 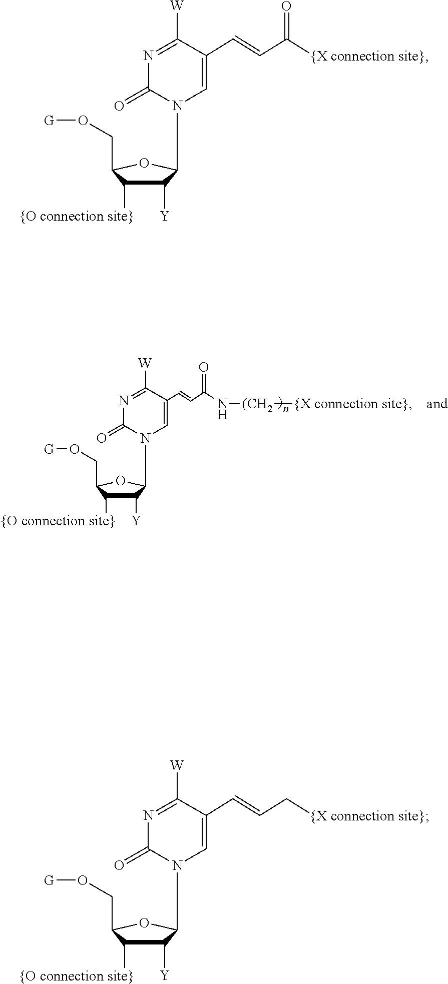
wherein n is 2-6, m is 2-3, Y is H, O-TBS, O—POM, or O-TOM, G is DMT or H, and W is OH, N═CHN(CH₃)₂, NHCOPh, or NHCOCH₃;
wherein -L- is present when Z is H.
